(19) United States
(12) Patent Application Publication
(10) Pub. No.: US 2010/0240580 A1
(43) Pub. Date: Sep. 23, 2010

(54) AZOLOARINE DERIVATIVES, METHOD FOR THE PRODUCTION THEREOF, PHARMACEUTICALS CONTAINING THESE COMPOUNDS AND THE USE THEREOF

(75) Inventors: Gerhard ZOLLER, Schoneck (DE); Marc Dietrich VOSS, Kronberg (DE); Hans MATTER, Langenselbold (DE); Andreas HERLING, Bad Camberg (DE); Christophe Philippo, Gif Sur Yvette (FR); Claudie NAMANE, Villiers-sur-Marne (FR); Juan-Antonio SANCHEZ-ARIAS, Madrid (ES)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/708,179

(22) Filed: Feb. 18, 2010

Related U.S. Application Data
(63) Continuation of application No. PCT/EP2008/006685, filed on Aug. 14, 2008.

(30) Foreign Application Priority Data
Aug. 23, 2007 (EP) 07291036.7
Feb. 25, 2008 (EP) 08290181.0

(51) Int. Cl. A61K 38/28; C07D 235/18; A61K 31/4184; A61P 3/10; A61P 3/04; A61P 5/50; A61P 25/28; A61P 9/00; A61P 3/06; A61P 25/18; C07D 401/12; A61K 31/4439; C07D 409/12; C07D 417/12; A61K 31/427; A61K 38/27; A61K 31/48; A61P 5/06

(52) U.S. Cl. 514/6.5; 548/310.7; 514/394; 546/273.4; 514/338; 548/304.7; 548/204; 514/365; 514/11.3; 514/250

(57) ABSTRACT

The invention relates to azoloarine derivatives of formula I

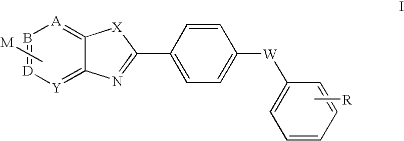

wherein R, A, B, D, Y, X, M and W are as defined herein, and their physiologically tolerated salts.

This application is a Continuation of International Application No. PCT/EP2008/006685, filed Aug. 14, 2008, which is incorporated herein by reference in its entirety.

The invention relates to azoloarine derivatives and their physiologically tolerated salts.

WO 99/11627 discloses phenyl benzimidazoles having antibacterial effect.

WO 03/032984 discloses aryl benzimidazoles as checkpoint kinase inhibitors. US 2006/019965 discloses benzimidazoles as gonadotropin releasing hormone receptor antagonists.

The invention was based on the object of providing novel compounds which display a therapeutically useful effect. The object was in particular to find novel compounds suitable for treating elevated lipid concentrations in the blood and in tissues, the metabolic syndrome, obesity, especially visceral (abdominal) obesity, including prevention of the sequelae associated therewith, diabetes, insulin resistance, deregulation of LDL, HDL and VLDL or cardiovascular disorders.

The invention therefore relates to compounds of the formula I,

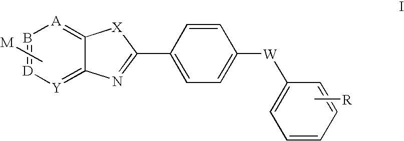

in which the meanings are
M R1-N(R2)-C(═O)—, R5-C(═O)—N(R1)-, R5-S(O)₀₋₂—N(R1)-, R1-N(R2)-S(O)₀₋₂—;
W O, CH₂, C═O;
X N—R4, O, S;
A, B, D, Y independently of one another C(R3) or N,
   where a maximum of two of the radicals A, B, D, Y can have the meaning of N;
R(C₁-C₁₆)-alkyl, (C₁-C₅)-alkyloxy, (C₁-C₅)-alkylthio, amino, (C₁-C₁₅)-alkylamino, di-(C₂-C₈)-alkylamino, (C₁-C₆)-alkylcarbonylamino, (C₁-C₆)-alkoxycarbonylamino, halogen, hydroxy, mono-(C₁-C₆)-alkylaminocarbonyl, di-(C₂-C₈)-alkylamino carbonyl, (C₁-C₆)-alkoxycarbonyl, (C₁-C₁₆)-alkylcarbonyl, cyano, trifluoromethyl, trifluoromethyloxy, (C₁-C₆)-alkylsulfonyl or aminosulfonyl;
R1 (C₂-C₁₀)-alkyl,
   where alkyl may be substituted by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino, —(C₆-C₁₀)-aryl, —(C₃-C₁₂)-heteroaryl, —(C₃-C₁₂)-heterocyclyl or —(C₃-C₁₂)-cycloalkyl,
      where aryl, heteroaryl, heterocyclyl or cycloalkyl may optionally be substituted one or more times by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino,
   (C₁-C₁₀)-alkyl,
      where alkyl is substituted by —(C₆-C₁₀)-aryl, —(C₃-C₁₂)-heteroaryl, —(C₃-C₁₂)-heterocyclyl or —(C₃-C₁₂)-cycloalkyl,
         where aryl, heteroaryl, heterocyclyl or cycloalkyl may optionally be substituted one or more times by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino;
   —(C₆-C₁₀)-aryl, —(C₃-C₁₂)-hetero aryl, —(C₃-C₁₂)-heterocyclyl or —(C₃-C₁₂)-cycloalkyl,
      where aryl, heteroaryl, heterocyclyl or cycloalkyl may optionally be substituted one or more times by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino;
R2 hydrogen, (C₂-C₁₆)-alkyl, —(C₆-C₁₀)-aryl, (C₁-C₄)-alkylene-(C₆-C₁₀)-aryl;
or R1 and R2 form together with the nitrogen atom carrying them a monocyclic, saturated or partly unsaturated 4- to 7-membered ring system or a bicyclic saturated or partly unsaturated 8- to 14 membered ring system whose individual members of the ring system may be replaced by one to three atoms or atomic groups from the group of —CHR4-, —CR4R5-, —(C═R4)-, —NR6-, —C(═O)—, —O—, —S—, —SO— and —SO₂—;
R3 hydrogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino, cyano, (C₁-C₆)-alkylcarbonyl, halogen, trifluoromethyl, trifluoromethyloxy, (C₁-C₆)-alkylsulfonyl, aminosulfonyl;
R4 hydrogen, (C₁-C₅)-alkyl;
R5 hydrogen, (C₁-C₁₆)-alkyl, amino, (C₁-C₁₆)-alkylamino, di-(C₂-C₁₂)-alkylamino,
   where alkyl may be substituted by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino, —(C₆-C₁₀)-aryl, —(C₃-C₁₂)-heteroaryl, —(C₃-C₁₂)-heterocyclyl or —(C₃-C₁₂)-cycloalkyl,
      where aryl, heteroaryl, heterocyclyl or cycloalkyl may optionally be substituted one or more times by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino;
   —(C₆-C₁₀)-aryl, —(C₃-C₁₂)-heteroaryl, —(C₃-C₁₂)-heterocyclyl or —(C₃-C₁₂)-cycloalkyl,
      where aryl, heteroaryl, heterocyclyl or cycloalkyl may optionally be substituted one or more times by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino;
         and the physiologically tolerated salts thereof.

Preference is given to compounds of the formula I, with
M R1-N(R2)-C(═O)—, R5-C(═O)—N(R1)-, R5-S(O)₀₋₂—N(R1)-, R1-N(R2)-S(O)₀₋₂—;
W O, C═O;
X N—R4;
A, B, D, Y independently of one another C(R3) or N,
   where a maximum of two of the radicals A, B, D, Y may have the meaning of N;
R (C₁-C₁₆)-alkyl, (C₁-C₅)-alkyloxy, (C₁-C₅)-alkylthio, amino, (C₁-C₅)-alkylamino, di-(C₂-C₈)-alkylamino, (C₁-C₆)-alkylcarbonylamino, (C₁-C₆)-alkoxycarbonylamino, halogen, hydroxy, mono-(C₁-C₆)-alkylaminocarbonyl, di-(C₂-C₈)-alkylaminocarbonyl, (C₁-C₆)-alkoxycarbonyl, (C₁-C₆)-alkylcarbonyl, cyano, trifluoromethyl, trifluoromethyloxy, (C₁-C₆)-alkylsulfonyl or aminosulfonyl;
R1 (C₂-C₁₀)-alkyl,
   where alkyl may be substituted by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino, —(C₆-C₁₀)-aryl, —(C₃-C₁₂)-heteroaryl, —(C₃-C₁₂)-heterocyclyl or —(C₃-C₁₂)-cycloalkyl,
      where aryl, heteroaryl, heterocyclyl or cycloalkyl may optionally be substituted one or more times by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino;
   (C₁-C₁₀)-alkyl,
      where alkyl is substituted by —(C₆-C₁₀)-aryl, —(C₃-C₁₂)-heteroaryl, (C₃-C₁₂)-heterocyclyl or —(C₃-C₁₂)-cycloalkyl,
         where aryl, heteroaryl, heterocyclyl or cycloalkyl may optionally be substituted one or more times by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino;
R2 hydrogen, (C₂-C₁₆)-alkyl, —(C₆-C₁₀)-aryl, (C₁-C₄)-alkylene-(C₆-C₁₀)-aryl;
or R1 and R2 form together with the nitrogen atom carrying them a monocyclic, saturated or partly unsaturated 4- to 7-membered ring system or a bicyclic saturated or partly unsaturated 8- to 14 membered ring system whose individual members of the ring systems may be replaced by one to three atoms or atomic groups from the group of —CHR4-CR4R5-, —(C═R4)-, —NR6-, —C(═O)—, —O—, —S—, —SO— and —SO₂—;
R3 hydrogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino, cyano, (C₁-C₆)-alkylcarbonyl, halogen, trifluoromethyl, trifluoromethyloxy, (C₁-C₆)-alkylsulfonyl, aminosulfonyl;
R4 hydrogen, (C₁-C₅)-alkyl
R5 hydrogen, (C₁-C₁₆)-alkyl, amino, (C₁-C₁₆)-alkylamino, di-(C₂-C₁₂)-alkylamino, where alkyl may be substituted by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino, —(C₆-C₁₀)-aryl, —(C₃-C₁₂)-heteroaryl, —(C₃-C₁₂)-heterocyclyl or —(C₃-C₁₂)-cycloalkyl,
   where aryl, heteroaryl, heterocyclyl or cycloalkyl may optionally be substituted one or more times by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino;
      and the physiologically tolerated salts thereof.

Particular preference is given to compounds of the formula I with R1-N(R2)-C(═O)—, R5-C(═O)—N(R1)-;
W O, C═O;
X NH;
A, B, D, Y CH;
R(C₁-C₁₆)-alkyl, (C₁-C₅)-alkyloxy, (C₁-C₅)-alkylthio, amino, (C₁-C₅)-alkylamino, di-(C₂-C₈)-alkylamino, (C₁-C₆)-alkylcarbonylamino, (C₁-C₆)-alkoxycarbonylamino, halogen, hydroxy, mono-(C₁-C₆)-alkylaminocarbonyl, di-(C₂-C₈)-alkylamino carbonyl, (C₁-C₆)-alkoxycarbonyl, (C₁-C₆)-alkylcarbonyl, cyano, trifluoromethyl, trifluoromethyloxy, (C₁-C₆)-alkylsulfonyl or aminosulfonyl;
R1 (C₁-C₁₀)-alkyl,
   where alkyl is substituted by —(C₆-C₁₀)-aryl, —(C₃-C₁₂)-heteroaryl, —(C₃-C₁₂)-heterocyclyl or —(C₃-C₁₂)-cycloalkyl,
      where aryl, heteroaryl, heterocyclyl or cycloalkyl may optionally be substituted one or more times by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino;
R2 hydrogen, (C₂-C₁₆)-alkyl, —(C₆-C₁₀)-aryl, (C₁-C₄)-alkylene-(C₆-C₁₀)-aryl;
R5 hydrogen, (C₁-C₁₆)-alkyl, amino, (C₁-C₁₆)-alkylamino, di-(C₂-C₁₂)-alkylamino,
   where alkyl is substituted by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino, —(C₆-C₁₀)-aryl, —(C₃-C₁₂)-heteroaryl, —(C₃-C₁₂)-heterocyclyl or —(C₃-C₁₂)-cycloalkyl,
      where aryl, heteroaryl, heterocyclyl or cycloalkyl may optionally be substituted one or more times by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino;
         and the physiologically tolerated salts thereof.

Very particular preference is given to compounds of the formula I with,
M R1-NH—C(═O)—;
W O, C═O;
X NH;
A, B, D, Y CH;
(C₁-C₁₆)-alkyl, (C₁-C₅)-alkyloxy, (C₁-C₅)-alkylthio, amino, (C₁-C₅)-alkylamino, di-(C₂-C₈)-alkylamino, (C₁-C₆)-alkylcarbonylamino, (C₁-C₆)-alkoxycarbonylamino, halogen, hydroxy, mono-(C₁-C₆)-alkylaminocarbonyl, di-(C₂-C₈)-alkylamino carbonyl, (C₁-C₆)-alkoxycarbonyl, (C₁-C₁₆)-alkylcarbonyl, cyano, trifluoromethyl, trifluoromethyloxy, (C₁-C₆)-alkylsulfonyl or aminosulfonyl;
R1 (C₁-C₁₀)-alkyl,
   where alkyl is substituted by —(C₆-C₁₀)-aryl, —(C₃-C₁₂)-heteroaryl, (C₃-C₁₂)-heterocyclyl or —(C₃-C₁₂)-cycloalkyl,
      where aryl, heteroaryl, heterocyclyl or cycloalkyl may optionally be substituted one or more times by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino;
         and the physiologically tolerated salts thereof.

Very particular preference is further given to compounds of the formula I with,
M R1-NH—C(═O)—;
W O, C═O;
X NH;
A, B, D, Y CH;
R (C₁-C₁₆)-alkyl, (C₁-C₅)-alkyloxy, (C₁-C₅)-alkylthio, amino, (C₁-C₅)-alkylamino, di-(C₂-C₈)-alkylamino, (C₁-C₆)-alkylcarbonylamino, (C₁-C₆)-alkoxycarbonylamino, halogen, hydroxy, mono-(C₁-C₆)-alkylaminocarbonyl, di-(C₂-C₈)-alkylamino carbonyl, (C₁-C₆)-alkoxycarbonyl, (C₁-C₆)-alkylcarbonyl, cyano, trifluoromethyl, trifluoromethyloxy, (C₁-C₆)-alkylsulfonyl or aminosulfonyl;
R1 —(CH₂)ₙ—(C₆-C₁₀)-aryl, —(CH₂)ₙ—(C₃-C₁₂)-heteroaryl, —(CH₂)ₙ—(C₃-C₁₂)-heterocyclyl or —(CH₂)ₙ—(C₃-C₁₂)-cycloalkyl,
   where aryl, heteroaryl, heterocyclyl or cycloalkyl rings may optionally be substituted one or more times by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino;
n 1, 2, 3, 4;
   and the physiologically tolerated salts thereof.

Compounds of the formula I preferred in one embodiment are those in which W is O.

Compounds of the formula I preferred in one embodiment are those in which W is CH₂.

Compounds of the formula I preferred in one embodiment are those in which W is C═O.

Compounds of the formula I preferred in one embodiment are those in which X is N—R4.

Compounds of the formula I preferred in one embodiment are those in which X is O.

Compounds of the formula I preferred in one embodiment are those in which X is S.

Compounds of the formula I preferred in one embodiment are those in which R1 is:
R1 (C₂-C₁₀)-alkyl,
   where alkyl may be substituted by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino, —(C₆-C₁₀)-aryl, —(C₃-C₁₂)-heteroaryl, —(C₃-C₁₂)-heterocyclyl or —(C₃-C₁₂)-cycloalkyl,
      where aryl, heteroaryl, heterocyclyl or cycloalkyl may optionally be substituted one or more times by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino.

Compounds of the formula I preferred in one embodiment are those in which R1 is:
(C₁-C₁₀)-alkyl,
   where alkyl is substituted by —(C₆-C₁₀)-aryl, —(C₃-C₁₂)-heteroaryl, —(C₃-C₁₂)-heterocyclyl or —(C₃-C₁₂)-cycloalkyl,
      where aryl, heteroaryl, heterocyclyl or cycloalkyl may optionally be substituted one or more times by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino.

Compounds of the formula I preferred in one embodiment are those in which A, B, D, Y are C(R3), C(R3), C(R3), C(R3).

Compounds of the formula I preferred in one embodiment are those in which A, B, D, Y are NH, C(R3), C(R3), C(R3).

Compounds of the formula I preferred in one embodiment are those in which A, B, D, Y are C(R3), NH, C(R3), C(R3).

Compounds of the formula I preferred in one embodiment are those in which A, B, D, Y are C(R3), C(R3), NH, C(R3).

Compounds of the formula I preferred in one embodiment are those in which A, B, D, Y are C(R3), C(R3), C(R3), NH.

Compounds of the formula I preferred in one embodiment are those in which A, B, D, Y are NH, NH, C(R3), C(R3).

Compounds of the formula I preferred in one embodiment are those in which A, B, D, Y are C(R3), NH, NH, C(R3).

Compounds of the formula I preferred in one embodiment are those in which A, B, D, Y are C(R3), C(R3), NH, NH.

Compounds of the formula I preferred in one embodiment are those in which A, B, D, Y are NH, C(R3), NH, C(R3).

Compounds of the formula I preferred in one embodiment are those in which A, B, D, Y are NH, C(R3), C(R3), NH.

Compounds of the formula I preferred in one embodiment are those in which A, B, D, Y are C(R3), NH, C(R3), NH.

Compounds of the formula I preferred in one embodiment are those in which R3 is hydrogen.

Compounds of the formula I preferred in one embodiment are those in which R3 is (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino, cyano, (C₁-C₆)-alkylcarbonyl, halogen, trifluoromethyl, trifluoro-methyloxy, (C₁-C₆)-alkylsulfonyl or aminosulfonyl.

Compounds of the formula I preferred in one embodiment are those in which R is hydrogen.

Compounds of the formula I preferred in one embodiment are those in which R is Cl.

Compounds of the formula I preferred in one embodiment are those in which R is CF₃.

Compounds of the formula I preferred in one embodiment are those in which M is R5-S(O)₀₋₂—N(R1)- or R1-N(R2)-S(O)₀₋₂—.

Compounds of the formula I preferred in one embodiment are those in which M is a heterocycle which may comprise 2 to 4 heteroatoms from the group of N, O, S, where the heterocycle may be substituted by (C₁-C₁₆)-alkyl or doubly bonded oxygen.

Compounds of the formula I preferred in one embodiment are those in which M is R1-N(R2)-C(═O)—.

Compounds of the formula I preferred in one embodiment are those in which M is R5-C(═O)—N(R1)-.

The invention further relates to the use of the compounds of the formula I

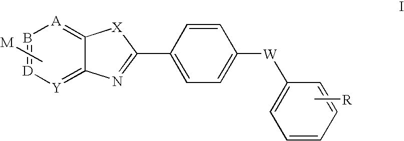

in which the meanings are
M R1-N(R2)-C(═O)—, R5-C(═O)—N(R1)-, R5-S(O)₀₋₂—N(R1)-, R1-N(R2)-S(O)₀₋₂—, a heterocycle which may comprise 2 to 4 heteroatoms from the group of N, O, S, where the heterocycle may be substituted by (C₁-C₁₆)-alkyl, or doubly bonded oxygen;
W O, CH₂, C═O;
X N—R4, O, S;
A, B, D, Y independently of one another C(R3) or N,
   where a maximum of two of the radicals A, B, D, Y can have the meaning of N;
R hydrogen, (C₁-C₁₆)-alkyl, (C₁-C₅)-alkyloxy, (C₁-C₅)-alkylthio, amino, (C₁-C₅)-alkylamino, di-(C₂-C₈)-alkylamino, (C₁-C₆)-alkylcarbonylamino, (C₁-C₆)-alkoxycarbonylamino, halogen, hydroxy, mono-(C₁-C₆)-alkylaminocarbonyl, di-(C₂-C₈)-alkylaminocarbonyl, (C₁-C₆)-alkoxycarbonyl, (C₁-C₆)-alkylcarbonyl, cyano, trifluoromethyl, trifluoromethyloxy, (C₁-C₆)-alkylsulfonyl or aminosulfonyl;
R1 (C₂-C₁₀)-alkyl,
   where alkyl may be substituted by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino, —(C₆-C₁₀)-aryl, —(C₃-C₁₂)-heteroaryl, —(C₃-C₁₂)-heterocyclyl or —(C₃-C₁₂)-cycloalkyl,
      where aryl, heteroaryl, heterocyclyl or cycloalkyl may optionally be substituted one or more times by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino,
   (C₁-C₁₀)-alkyl,
      where alkyl is substituted by —(C₆-C₁₀)-aryl, —(C₃-C₁₂)-heteroaryl, —(C₃-C₁₂)-heterocyclyl or —(C₃-C₁₂)-cycloalkyl,
         where aryl, heteroaryl, heterocyclyl or cycloalkyl may optionally be substituted one or more times by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino;
   —(C₆-C₁₀)-aryl, —(C₃-C₁₂)-heteroaryl, —(C₃-C₁₂)-heterocyclyl or —(C₃-C₁₂)-cycloalkyl,
      where aryl, heteroaryl, heterocyclyl or cycloalkyl may optionally be substituted one or more times by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino;
R2 hydrogen, (C₂-C₁₆)-alkyl, —(C₆-C₁₀)-aryl, (C₁-C₄)-alkylene-(C₆-C₁₀)-aryl;
or R1 and R2 form together with the nitrogen atom carrying them a monocyclic, saturated or partly unsaturated 4- to 7-membered ring system or a bicyclic saturated or partly unsaturated 8- to 14 membered ring system whose individual members of the ring systems may be replaced by one to three atoms or atomic groups from the group of —CHR4-, —CR4R5-, —(C═R4)-, —NR6-, —C(═O)—, —O—, —S—, —SO— and —SO₂—;
R3 hydrogen, (C₁-C₆)-alkyl, (C₁-C₃)-alkyloxy, hydroxy, (C₁-C₁₆)-alkylmercapto, amino, (C₁-C₆)-alkylamino, di-(C₂-C₁₂)-alkylamino, cyano, (C₁-C₆)-alkylcarbonyl, halogen, trifluoromethyl, trifluoromethyloxy, (C₁-C₆)-alkylsulfonyl, aminosulfonyl;
R4 hydrogen, (C₁-C₅)-alkyl;
R5 hydrogen, (C₁-C₁₆)-alkyl, amino, (C₁-C₁₆)-alkylamino, di-(C₂-C₁₂)-alkylamino,
   where alkyl may be substituted by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino, —(C₆-C₁₀)-aryl, —(C₃-C₁₂)-heteroaryl, —(C₃-C₁₂)-heterocyclyl or —(C₃-C₁₂)-cycloalkyl,
      where aryl, heteroaryl, heterocyclyl or cycloalkyl may optionally be substituted one or more times by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino;
   —(C₆-C₁₀)-aryl, —(C₃-C₁₂)-heteroaryl, —(C₃-C₁₂)-heterocyclyl or —(C₃-C₁₂)-cycloalkyl,
      where aryl, heteroaryl, heterocyclyl or cycloalkyl may optionally be substituted one or more times by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino;
         and the physiologically tolerated salts thereof, for the manufacture of a medicament for the treatment of obesity.

Preference is given to the use of the compounds of the formula I in which the meanings are
M R1-N(R2)-C(═O)—, R5-C(═O)—N(R1)-, R5-S(O)₀₋₂—N(R1)-, R1-N(R2)-S(O)₀₋₂—, a heterocycle which may comprise 2 to 4 heteroatoms from the group of N, O, S, where the heterocycle may be substituted by (C₁-C₁₆)-alkyl, or doubly bonded oxygen;
W O, C═O;
X N—R4;
A, B, D, Y independently of one another C(R3) or N,
   where a maximum of two of the radicals A, B, D, Y may have the meaning of N;
R hydrogen, (C₁-C₁₆)-alkyl, (C₁-C₅)-alkyloxy, (C₁-C₅)-alkylthio, amino, (C₁-C₁₅)-alkylamino, di-(C₂-C₈)-alkylamino, (C₁-C₆)-alkylcarbonylamino, (C₁-C₆)-alkoxycarbonylamino, halogen, hydroxy, mono-(C₁-C₆)-alkylaminocarbonyl, di-(C₂-C₈)-alkylamino carbonyl, (C₁-C₆)-alkoxycarbonyl, (C₁-C₁₆)-alkylcarbonyl, cyano, trifluoromethyl, trifluoromethyloxy, (C₁-C₆)-alkylsulfonyl or aminosulfonyl;
R1 (C₂-C₁₀)-alkyl,
   where alkyl may be substituted by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino, —(C₆-C₁₀)-aryl, —(C₃-C₁₂)-heteroaryl, —(C₃-C₁₂)-heterocyclyl or —(C₃-C₁₂)-cycloalkyl,
      where aryl, heteroaryl, heterocyclyl or cycloalkyl may optionally be substituted one or more times by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino;
   (C₁-C₁₀)-alkyl,
      where alkyl is substituted by —(C₆-C₁₀)-aryl, —(C₃-C₁₂)-heteroaryl, —(C₃-C₁₂)-heterocyclyl or —(C₃-C₁₂)-cycloalkyl,
         where aryl, heteroaryl, heterocyclyl or cycloalkyl may optionally be substituted one or more times by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino;
R2 hydrogen, (C₂-C₁₆)-alkyl, —(C₆-C₁₀)-aryl, (C₁-C₄)-alkylene-(C₆-C₁₀)-aryl;
or R1 and R2 form together with the nitrogen atom carrying them a monocyclic, saturated or partly unsaturated 4- to 7-membered ring system or a bicyclic saturated or partly unsaturated 8- to 14 membered ring system whose individual members of the ring systems may be replaced by one to three atoms or atomic groups from the group of —CHR4-, —CR4R5-, —(C═R4)-, —NR6-, —C(═O)—, —O—, —S—, —SO— and —SO₂—;
R3 hydrogen, (C₁-C₆)-alkyl, (C₁-C₃)-alkyloxy, hydroxy, (C₁-C₁₆)-alkylmercapto, amino, (C₁-C₆)-alkylamino, di-(C₂-C₁₂)-alkylamino, cyano, (C₁-C₆)-alkylcarbonyl, halogen, trifluoromethyl, trifluoromethyloxy, (C₁-C₆)-alkylsulfonyl, aminosulfonyl;
R4 hydrogen, (C₁-C₅)-alkyl
R5 hydrogen, (C₁-C₁₆)-alkyl, amino, (C₁-C₁₆)-alkylamino, di-(C₂-C₁₂)-alkylamino, where alkyl may be substituted by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino, (C₆-C₁₀)-aryl, —(C₃-C₁₂)-heteroaryl, —(C₃-C₁₂)-heterocyclyl or —(C₃-C₁₂)-cycloalkyl,
   where aryl, heteroaryl, heterocyclyl or cycloalkyl may optionally be substituted one or more times by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino;
      and the physiologically tolerated salts thereof, for the manufacture of a medicament for the treatment of obesity.

Particular preference is given to the use of the compounds of the formula I in which the meanings are
M R1-N(R2)-C(═O)—, R5-C(═O)—N(R1)-;
W O, C═O;
X N—R4;
A, B, D, Y independently of one another C(R3) or N,
   where a maximum of two of the radicals A, B, D, Y may have the meaning of N;
R hydrogen, (C₁-C₁₆)-alkyl, (C₁-C₅)-alkyloxy, (C₁-C₅)-alkylthio, amino, (C₁-C₁₅)-alkylamino, di-(C₂-C₈)-alkylamino, (C₁-C₆)-alkylcarbonylamino, (C₁-C₆)-alkoxycarbonylamino, halogen, hydroxy, mono-(C₁-C₆)-alkylaminocarbonyl, di-(C₂-C₈)-alkylamino carbonyl, (C₁-C₆)-alkoxycarbonyl, (C₁-C₁₆)-alkylcarbonyl, cyano, trifluoromethyl, trifluoromethyloxy, (C₁-C₆)-alkylsulfonyl or aminosulfonyl;
R1 (C₂-C₁₀)-alkyl,
   where alkyl may be substituted by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino, —(C₆-C₁₀)-aryl, —(C₃-C₁₂)-heteroaryl, —(C₃-C₁₂)-heterocyclyl or —(C₃-C₁₂)-cycloalkyl,
      where aryl, heteroaryl, heterocyclyl or cycloalkyl may optionally be substituted one or more times by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino;
   (C₁-C₁₀)-alkyl,
      where alkyl is substituted by —(C₆-C₁₀)-aryl, —(C₃-C₁₂)-heteroaryl, —(C₃-C₁₂)-heterocyclyl or —(C₃-C₁₂)-cycloalkyl,
         where aryl, heteroaryl, heterocyclyl or cycloalkyl may optionally be substituted one or more times by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino;
R2 hydrogen, (C₂-C₁₆)-alkyl, —(C₆-C₁₀)-aryl, (C₁-C₄)-alkylene-(C₆-C₁₀)-aryl;
or R1 and R2 form together with the nitrogen atom carrying them a monocyclic, saturated or partly unsaturated 4- to 7-membered ring system or a bicyclic saturated or partly unsaturated 8- to 14 membered ring system whose individual members of the ring systems may be replaced by one to three atoms or atomic groups from the group of —CHR4-, —CR4R5-, —(C═R4)-, —NR6-, —C(═O)—, —O—, —S—, —SO— and —SO₂—;
R3 hydrogen, (C₁-C₆)-alkyl, (C₁-C₃)-alkyloxy, hydroxy, (C₁-C₁₆)-alkylmercapto, amino, (C₁-C₆)-alkylamino, di-(C₂-C₁₂)-alkylamino, cyano, (C₁-C₆)-alkylcarbonyl, halogen, trifluoromethyl, trifluoromethyloxy, (C₁-C₆)-alkylsulfonyl, aminosulfonyl;
R4 hydrogen, (C₁-C₅)-alkyl
R5 hydrogen, (C₁-C₁₆)-alkyl, amino, (C₁-C₁₆)-alkylamino, di-(C₂-C₁₂)-alkylamino, where alkyl may be substituted by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino, (C₆-C₁₀)-aryl, —(C₃-C₁₂)-heteroaryl, —(C₃-C₁₂)-heterocyclyl or —(C₃-C₁₂)-cycloalkyl,
   where aryl, heteroaryl, heterocyclyl or cycloalkyl may optionally be substituted one or more times by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino;
      and the physiologically tolerated salts thereof, for the manufacture of a medicament for the treatment of obesity.

Very particular preference is given to the use of the compounds of the formula I in which the meanings are
M R1-N(R2)-C(═O)—, R5-C(═O)—N(R1)-;
W O, C═O;
X N—R4;
A, B, D, Y CH;
R hydrogen, (C₁-C₁₆)-alkyl, (C₁-C₅)-alkyloxy, (C₁-C₅)-alkylthio, amino, (C₁-C₅)-alkylamino, di-(C₂-C₈)-alkylamino, (C₁-C₆)-alkylcarbonylamino, (C₁-C₆)-alkoxycarbonylamino, halogen, hydroxy, mono-(C₁-C₆)-alkylaminocarbonyl, di-(C₂-C₈)-alkylamino carbonyl, (C₁-C₆)-alkoxycarbonyl, (C₁-C₆)-alkylcarbonyl, cyano, trifluoromethyl, trifluoromethyloxy, (C₁-C₆)-alkylsulfonyl or aminosulfonyl;
R1 (C₂-C₁₀)-alkyl,
   where alkyl may be substituted by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino, —(C₆-C₁₀)-aryl, —(C₃-C₁₂)-heteroaryl, —(C₃-C₁₂)-heterocyclyl or —(C₃-C₁₂)-cycloalkyl,
      where aryl, heteroaryl, heterocyclyl or cycloalkyl may optionally be substituted one or more times by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino;
   (C₁-C₁₀)-alkyl,
      where alkyl is substituted by —(C₆-C₁₀)-aryl, —(C₃-C₁₂)-heteroaryl, —(C₃-C₁₂)-heterocyclyl or —(C₃-C₁₂)-cycloalkyl,
         where aryl, heteroaryl, heterocyclyl or cycloalkyl may optionally be substituted one or more times by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino;
R2 hydrogen, (C₂-C₁₆)-alkyl, —(C₆-C₁₀)-aryl, (C₁-C₄)-alkylene-(C₆-C₁₀)-aryl;
   or R1 and R2 form together with the nitrogen atom carrying them a monocyclic, saturated or partly unsaturated 4- to 7-membered ring system or a bicyclic saturated or partly unsaturated 8- to 14 membered ring system whose individual members of the ring systems may be replaced by one to three atoms or atomic groups from the group of —CHR4-, —CR4R5-, —(C═R4)-, —NR6-, —C(═O)—, —O—, —S—, —SO— and —SO₂—;
R4 hydrogen;
R5 hydrogen, (C₁-C₁₆)-alkyl, amino, (C₁-C₁₆)-alkylamino, di-(C₂-C₁₂)-alkylamino
   where alkyl may be substituted by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino, —(C₆-C₁₀)-aryl, —(C₃-C₁₂)-heteroaryl, —(C₃-C₁₂)-heterocyclyl or —(C₃-C₁₂)-cycloalkyl,
      where aryl, heteroaryl, heterocyclyl or cycloalkyl may optionally be substituted one or more times by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino;
         and the physiologically tolerated salts thereof for the manufacture of a medicament for the treatment of obesity.

The invention relates to compounds of the formula I in the form of their salts, racemates, optical isomers, rotational isomers, racemic mixtures and pure enantiomers, and their diastereomers and mixtures thereof. The mixtures are separated by chromatographic means.

The present invention includes all possible tautomeric forms of the compounds of the formula I.

The present invention further includes derivatives of the compounds of the formula I, for example solvates such as hydrates and alcohol adducts, esters, prodrugs and other physiologically acceptable derivatives of the compounds of the formula I, and active metabolites of the compounds of the formula I. The invention likewise includes all crystal modifications of the compounds of the formula I.

The alkyl radicals in the substituents R, R1, R2, and R3 may be either straight-chain or branched.

Halogen is fluorine, chlorine, bromine or iodine, in particular bromine or chlorine.

Aryl is a monocyclic or bicyclic aromatic hydrocarbon radical having 6 to 10 ring atoms which may be substituted independently by one to four, preferably one or two, described substituents.

Heteroaryl is a monocyclic or bicyclic radical having 5 to 12 ring atoms having at least one aromatic ring having one, two or three ring heteroatoms selected from N, O and S, the remaining ring atoms being C.

Cycloalkyl is a saturated or partly unsaturated ring system (which comprises exclusively carbon atoms) which includes one or more rings.

Heterocyclyl is a saturated or partly unsaturated ring system (which comprises at least one heteroatom) which includes one or more rings.

Bicyclyl is a bicyclic saturated or partly unsaturated ring system, where the individual members of the ring systems may comprise exclusively carbon atoms or one, two or three ring heteroatoms selected from N, O and S, the remaining ring atoms being C. One of the rings in the bicyclic system may be a fused aromatic ring such as benzene.

If radicals or substituents occur more than once in the compounds of the formula I (such as, for example, “R3”), they may all have the stated meanings independently of one another and be identical or different.

Physiologically tolerated salts are, because their solubility in water is greater than that of the initial or basic compounds, particularly suitable for medical applications. These salts must have a physiologically tolerated anion or cation. Suitable physiologically tolerated acid addition salts of the compounds of the invention are salts of inorganic acids such as hydrochloric acid, hydrobromic, phosphoric, metaphosphoric, nitric, sulfamic and sulfuric acid, and of organic acids such as, for example, acetic acid, benzenesulfonic, benzoic, citric, ethanesulfonic, fumaric, gluconic, glycolic, isethionic, lactic, lactobionic, maleic, malic, methanesulfonic, succinic, p-toluenesulfonic and tartaric acid. The chlorine salt is particularly preferably used for medical purposes. Suitable physiologically tolerated basic salts are ammonium salts, alkali metal salts (such as sodium and potassium salts), alkaline earth metal salts (such as magnesium and calcium salts), zinc salts and salts of trometamol (2-amino-2-hydroxymethyl-1,3-propanediol), diethanolamine, lysine, arginine, choline, meglumine or ethylenediamine.

Salts with a physiologically nontolerated anion or cation likewise belong within the framework of the invention as useful intermediates for the preparation or purification of physiologically tolerated salts and/or for use in nontherapeutic, for example in vitro, applications.

A further aspect of this invention are prodrugs of the compounds of the invention. Such prodrugs can be metabolized in vivo to a compound of the invention. These prodrugs may themselves be active or not.

The compounds of the invention may also exist in various polymorphous forms, for example as amorphous and crystalline polymorphous forms. All polymorphous forms of the compounds of the invention belong within the framework of the invention and are a further aspect of the invention.

All references to “compound(s) of formula (I)” hereinafter refer to compound(s) of the formula (I) as described above, and their salts, solvates and physiologically functional derivatives as described herein.

The compounds of the formula I and the physiologically tolerated salts and physiologically functional derivatives thereof represent ideal medicaments for the treatment of elevated lipid concentrations in the blood and in tissues, the metabolic syndrome, obesity, diabetes, insulin resistance, deregulation of LDL, HDL and VLDL or cardiovascular disorders, lipid metabolism impairments, especially hyperlipidemia.

The compound(s) of the formula (I) can also be administered in combination with further active ingredients.

The amount of a compound of formula (I) necessary to achieve the desired biological effect depends on a number of factors, for example the specific compound chosen, the intended use, the mode of administration and the clinical condition of the patient. The daily dose is generally in the range from 0.1 mg to 100 mg (typically from 0.1 mg to 50 mg) per day and per kilogram of body weight, for example 0.1-10 mg/kg/day. Tablets or capsules may contain, for example, from 0.01 to 100 mg, typically from 0.02 to 50 mg. For the prophylaxis or therapy of the above-mentioned conditions, the compounds of formula (I) may be used as the compound itself, but they are preferably in the form of a pharmaceutical composition with an acceptable carrier. The carrier must, of course, be acceptable in the sense that it is compatible with the other ingredients of the composition and is not harmful for the patient's health. The carrier may be a solid or a liquid or both and is preferably formulated with the compound as a single dose, for example as a tablet, which may contain from 0.05% to 95% by weight of the active ingredient. Other pharmaceutically active substances may likewise be present, including other compounds of formula (I). The pharmaceutical compositions of the invention can be produced by one of the known pharmaceutical methods, which essentially consist of mixing the ingredients with pharmacologically acceptable carriers and/or excipients.

Pharmaceutical compositions of the invention are those suitable for oral and peroral (for example sublingual) administration, although the most suitable mode of administration depends in each individual case on the nature and severity of the condition to be treated and on the nature of the compound of formula (I) used in each case. Coated formulations and coated slow-release formulations also belong within the framework of the invention. Preference is given to acid- and gastric juice-resistant formulations. Suitable coatings resistant to gastric juice comprise cellulose acetate phthalate, polyvinyl acetate phthalate, hydroxypropylmethylcellulose phthalate and anionic polymers of methacrylic acid and methyl methacrylate.

Suitable pharmaceutical preparations for oral administration may be in the form of separate units such as, for example, capsules, cachets, suckable tablets or tablets, each of which contains a defined amount of at least one compound of formula (I); as powders or granules; as solution or suspension in an aqueous or nonaqueous liquid; or as an oil-in-water or water-in-oil emulsion. These compositions may, as already mentioned, be prepared by any suitable pharmaceutical method which includes a step in which the active ingredient and the carrier (which may consist of one or more additional ingredients) are brought into contact. The compositions are generally produced by uniform and homogeneous mixing of the active ingredient with a liquid and/or finely divided solid carrier, after which the product is shaped if necessary. Thus, for example, a tablet can be produced by compressing or molding a powder or granules of the compound, where appropriate with one or more additional ingredients. Compressed tablets can be produced by tableting the compound in free-flowing form such as, for example, a powder or granules, where appropriate mixed with a binder, glidant, inert diluent and/or one (or more) surface-active/dispersing agent(s) in a suitable machine. Molded tablets can be produced by molding the compound, which is in powder form and is moistened with an inert liquid diluent, in a suitable machine.

Pharmaceutical compositions which are suitable for peroral (sublingual) administration comprise suckable tablets which contain at least one compound of formula (I) with a flavoring, normally sucrose and gum arabic or tragacanth, and pastilles which comprise the compound in an inert base such as gelatin and glycerol or sucrose and gum arabic.

Combinations with Other Medicaments

The compounds of the invention can be administered alone or in combination with one or more further pharmacologically active substances which have, for example, beneficial effects on metabolic disturbances or disorders frequently associated therewith. Examples of such medicaments are
1. medicaments which lower blood glucose, antidiabetics,
2. active ingredients for the treatment of dyslipidemias,
3. antiatherosclerotic medicaments,
4. antiobesity agents,
5. antiinflammatory active ingredients
6. active ingredients for the treatment of malignant tumors
7. antithrombotic active ingredients
8. active ingredients for the treatment of high blood pressure
9. active ingredients for the treatment of heart failure and
10. active ingredients for the treatment and/or prevention of complications caused by diabetes or associated with diabetes.

Further active ingredients suitable for combination products are in particular:

All antidiabetics which are mentioned in the Rote Liste 2006, chapter 12; all weight-reducing agents/appetite suppressants which are mentioned in the Rote Liste 2005, chapter 1; all lipid-lowering agents which are mentioned in the Rote Liste 2006, chapter 58. They can be combined with the compounds of the invention of the formula I in particular for a synergistic improvement in the effect. Administration of the active ingredient combination can take place either by separate administration of the active ingredients to the patient or in the form of combination products in which a plurality of active ingredients are present in one pharmaceutical preparation. Most of the active ingredients mentioned hereinafter are disclosed in the USP Dictionary of USAN and International Drug Names, US Pharmacopeia, Rockville 2001.

Antidiabetics include insulin and insulin derivatives such as, for example, LANTUS (see www.lantus.com) or HMR 1964 or those described in WO2005005477 (Novo Nordisk), fast-acting insulins (see U.S. Pat. No. 6,221,633), inhalable insulins such as, for example, EXUBERA® or oral insulins such as, for example, IN-105 (Nobex) or ORAL-LYN™ (Generex Biotechnology), GLP-1-derivatives such as, for example, exenatide, liraglutide or those which have been disclosed in WO98/08871 or WO2005027978 of Novo Nordisk A/S, in WO01/04156 of Zealand or in WO00/34331 of Beaufour-Ipsen, pramlintide acetate (Symlin; Amylin Pharmaceuticals), and orally effective hypoglycemic active ingredients.

The orally effective hypoglycemic active ingredients include preferably sulfonylureas,

biguanides,
meglitinides,
oxadiazolidinediones,
thiazolidinediones,
glucosidase inhibitors,
inhibitors of glycogen phosphorylase,
glucagon antagonists,
glucokinase activators,
inhibitors of fructose 1,6-bisphosphatase
modulators of glucose transporter 4 (GLUT4),
inhibitors of glutamine-fructose-6-phosphate amidotransferase (GFAT),
GLP-1 agonists, potassium channel openers such as, for example, those which have been disclosed in WO 97/26265 and WO 99/03861 of Novo Nordisk A/S,
inhibitors of dipeptidylpeptidase IV (DPP-IV),
insulin sensitizers,
inhibitors of liver enzymes involved in stimulating gluconeogenesis and/or glycogenolysis,
modulators of glucose uptake, of glucose transport and of glucose reabsorption,
inhibitors of 11β-HSD1,
inhibitors of protein tyrosine phosphatase 1B (PTP1B),
modulators of the sodium-dependent glucose transporter 1 or 2 (SGLT1, SGLT2),
compounds which alter lipid metabolism such as antihyperlipidemic active ingredients and antilipidemic active ingredients,
compounds which reduce food intake,
compounds which increase thermogenesis,
PPAR and RXR modulators and
active ingredients which act on the ATP-dependent potassium channel of the beta cells.

In one embodiment of the invention, the compound of the formula I is administered in combination with an HMGCoA reductase inhibitor such as simvastatin, fluvastatin, pravastatin, lovastatin, atorvastatin, cerivastatin, rosuvastatin or L-659699

In one embodiment of the invention, the compound of the formula I is administered in combination with a cholesterol absorption inhibitor such as, for example, ezetimibe, tiqueside, pamaqueside, FM-VP4 (sitostanol/campesterol ascorbyl phosphate; Forbes Medi-Tech, WO2005042692), MD-0727 (Microbia Inc., WO2005021497) or with compounds as described in WO2002066464 (Kotobuki Pharmaceutical Co. Ltd.), WO2005062824 (Merck & Co.) or WO2005061451 and WO2005061452 (AstraZeneca AB).

In one embodiment of the invention, the compound of the formula I is administered in combination with a PPAR gamma agonist such as, for example, rosiglitazone, pioglitazone, JTT-501, G1262570, R-483 or CS-011 (rivoglitazone).

In one embodiment of the invention, the compound of the formula I is administered in combination with a PPAR alpha agonist such as, for example, GW9578, GW-590735, K-111, LY-674, KRP-101 or DRF-10945.

In one embodiment of the invention, the compound of the formula I is administered in combination with a mixed PPAR alpha/gamma agonist such as, for example, muraglitazar, tesaglitazar, naveglitazar, LY-510929, ONO-5129, E-3030, AVE 8042, AVE 8134, AVE 0847, or as described in PCT/US00/11833, PCT/US00/11490, DE10142734.4 or in J. P. Berger et al., TRENDS in Pharmacological Sciences 28(5), 244-251, 2005.

In one embodiment of the invention, the compound of the formula I is administered in combination with a PPAR delta agonist such as, for example, GW-501516.

In one embodiment, the compound of the formula I is administered in combination with metaglidasen or with MBX-2044 or other partial PPAR gamma agonists/antagonists.

In one embodiment of the invention, the compound of the formula I is administered in combination with a fibrate such as, for example, fenofibrate, clofibrate or bezafibrate.

In one embodiment of the invention, the compound of the formula I is administered in combination with an MTP inhibitor such as, for example, implitapide, BMS-201038, R-103757 or those described in WO2005085226.

In one embodiment of the invention, the compound of the formula I is administered in combination with a CETP inhibitor such as, for example, torcetrapib or JTT-705.

In one embodiment of the invention, the compound of the formula I is administered in combination with a bile acid absorption inhibitor (see, for example, U.S. Pat. No. 6,245,744, U.S. Pat. No. 6,221,897 or WO00/61568), such as, for example, HMR 1741 or those as described in DE 10 2005 033099.1 and DE 10 2005 033100.9.

In one embodiment of the invention, the compound of the formula I is administered in combination with a polymeric bile acid adsorbent such as, for example, cholestyramine or colesevelam.

In one embodiment of the invention, the compound of the formula I is administered in combination with an LDL receptor inducer (see U.S. Pat. No. 6,342,512), such as, for example, HMR1171, HMR1586 or those as described in WO2005097738.

In one embodiment, the compound of the formula I is administered in combination with Omacor® (omega-3 fatty acids; highly concentrated ethyl esters of eicosapentaenoic acid and of docosahexaenoic acid).

In one embodiment of the invention, the compound of the formula I is administered in combination with an ACAT inhibitor such as, for example, avasimibe.

In one embodiment of the invention, the compound of the formula I is administered in combination with an antioxidant such as, for example, OPC-14117, probucol, tocopherol, ascorbic acid, β-carotene or selenium.

In one embodiment of the invention, the compound of the formula I is administered in combination with a vitamin such as, for example, vitamin B6 or vitamin B12.

In one embodiment of the invention, the compound of the formula I is administered in combination with a lipoprotein lipase modulator such as, for example, ibrolipim (NO-1886).

In one embodiment of the invention, the compound of the formula I is administered in combination with an ATP citrate lyase inhibitor such as, for example, SB-204990.

In one embodiment of the invention, the compound of the formula I is administered in combination with a squalene synthetase inhibitor such as, for example, BMS-188494 or as described in WO2005077907.

In one embodiment of the invention, the compound of the formula I is administered in combination with a lipoprotein(a) antagonist such as, for example, gemcabene (CI-1027).

In one embodiment of the invention, the compound of the formula I is administered in combination with an HM74A receptor agonist such as, for example, nicotinic acid.

In one embodiment of the invention, the compound of the formula I is administered in combination with a lipase inhibitor such as, for example, orlistat or cetilistat (ATL-962).

In one embodiment of the invention, the compound of the formula I is administered in combination with insulin.

In one embodiment, the compound of the formula I is administered in combination with a sulfonylurea such as, for example, tolbutamide, glibenclamide, glipizide or glimepiride.

In one embodiment, the compound of the formula I is administered in combination with a biguanide such as, for example, metformin.

In another embodiment, the compound of the formula I is administered in combination with a meglitinide such as, for example, repaglinide or nateglinide.

In one embodiment, the compound of the formula I is administered in combination with a thiazolidinedione such as, for example, troglitazone, ciglitazone, pioglitazone, rosiglitazone or the compounds disclosed in WO 97/41097 of Dr. Reddy's Research Foundation, in particular 5-[[4-[(3,4-dihydro-3-methyl-4-oxo-2-quinazolinylmethoxy]phenyl]methyl]-2,4-thiazolidinedione.

In one embodiment, the compound of the formula I is administered in combination with an α-glucosidase inhibitor such as, for example, miglitol or acarbose.

In one embodiment, the compound of the formula I is administered in combination with an active ingredient which acts on the ATP-dependent potassium channel of the beta cells, such as, for example, tolbutamide, glibenclamide, glipizide, glimepiride or repaglinide.

In one embodiment, the compound of the formula I is administered in combination with more than one of the aforementioned compounds, e.g. in combination with a sulfonylurea and metformin, a sulfonylurea and acarbose, repaglinide and metformin, insulin and a sulfonylurea, insulin and metformin, insulin and troglitazone, insulin and lovastatin, etc.

In one embodiment, the compound of the formula I is administered in combination with an inhibitor of glycogen phosphorylase, such as, for example, PSN-357 or FR-258900 or those as described in WO2003084922, WO2004007455, WO2005073229-31 or WO2005067932.

In one embodiment, the compound of the formula I is administered in combination with glucagon receptor antagonists such as, for example, A-770077, NNC-25-2504 or as described in WO2004100875 or WO2005065680.

In one embodiment, the compound of the formula I is administered in combination with activators of glucokinase, such as, for example, LY-2121260 (WO2004063179), PSN-105, PSN-110, GKA-50 or those as are described for example in WO2004072031, WO2004072066 or WO2005080360.

In one embodiment, the compound of the formula I is administered in combination with an inhibitor of gluconeogenesis, such as, for example, FR-225654.

In one embodiment, the compound of the formula I is administered in combination with inhibitors of fructose-1,6-bisphosphatase (FBPase), such as, for example, CS-917.

In one embodiment, the compound of the formula I is administered in combination with modulators of glucose transporter 4 (GLUT4), such as, for example, KST-48 (D.-O. Lee et al.:

Arzneim.-Forsch. Drug Res. 54 (12), 835 (2004)).

In one embodiment, the compound of the formula I is administered in combination with inhibitors of glutamine-fructose-6-phosphate amidotransferase (GFAT), as are described for example in WO2004101528.

In one embodiment, the compound of the formula I is administered in combination with inhibitors of dipeptidylpeptidase IV (DPP-IV), such as, for example, vildagliptin (LAF-237), sitagliptin (MK-0431), saxagliptin (BMS-477118), GSK-823093, PSN-9301, SYR-322, SYR-619, TA-6666, TS-021, GRC-8200, GW-825964X or as are described in WO2003074500, WO2003106456, WO200450658, WO2005058901, WO2005012312, WO2005012308, PCT/EP2005/007821, PCT/EP2005/008005, PCT/EP2005/008002, PCT/EP2005/008004, PCT/EP2005/008283, DE 10 2005 012874.2 or DE 10 2005 012873.4.

In one embodiment, the compound of the formula I is administered in combination with inhibitors of 11-beta-hydroxysteroid dehydrogenase 1 (11β-HSD1), such as, for example, BVT-2733 or those as are described for example in WO200190090-94, WO200343999, WO2004112782, WO200344000, WO200344009, WO2004112779, WO2004113310, WO2004103980, WO2004112784, WO 2003065983, WO2003104207, WO2003104208, WO2004106294, WO2004011410, WO2004033427, WO2004041264, WO2004037251, WO2004056744, WO2004065351, WO2004089367, WO2004089380, WO2004089470-71, WO2004089896, WO2005016877 or WO2005097759.

In one embodiment, the compound of the formula I is administered in combination with inhibitors of protein tyrosine phosphatase 1B (PTP1B), as are described for example in WO200119830-31, WO200117516, WO2004506446, WO2005012295, PCT/EP2005/005311, PCT/EP2005/005321, PCT/EP2005/007151 or DE 10 2004 060542.4.

In one embodiment, the compound of the formula I is administered in combination with modulators of the sodium-dependent glucose transporter 1 or 2 (SGLT1, SGLT2), such as, for example, KGA-2727, T-1095, SGL-0010, AVE 2268 and SAR 7226 or as are described for example in WO2004007517, WO200452903, WO200452902, PCT/EP2005/005959, WO2005085237, JP2004359630 or by A. L. Handlon in Expert Opin. Ther. Patents (2005) 15(11), 1531-1540.

In one embodiment, the compound of the formula I is administered in combination with modulators of GPR40.

In one embodiment, the compound of the formula I is administered in combination with inhibitors of hormone-sensitive lipase (HSL) as described for example in WO2005073199.

In one embodiment, the compound of the formula I is administered in combination with inhibitors of acetyl-CoA carboxylase (ACC), such as, for example, those as described in WO199946262, WO200372197, WO2003072197 or WO2005044814.

In one embodiment, the compound of the formula I is administered in combination with an inhibitor of phosphoenolpyruvate carboxykinase (PEPCK), such as, for example, those as described in WO2004074288.

In one embodiment, the compound of the formula I is administered in combination with an inhibitor of glycogen synthase kinase 3 beta (GSK-3 beta), as described for example in US2005222220, WO2005085230, PCT/EP2005/005346, WO2003078403, WO2004022544, WO2003106410, WO2005058908, US2005038023, WO2005009997, US2005026984, WO2005000836, WO2004106343, EP1460075, WO2004014910, WO2003076442, WO2005087727 or WO2004046117.

In one embodiment, the compound of the formula I is administered in combination with an inhibitor of protein kinase C beta (PKC beta), such as, for example, ruboxistaurin.

In one embodiment, the compound of the formula I is administered in combination with an endothelin A receptor antagonist such as, for example, avosentan (SPP-301).

In one embodiment, the compound of the formula I is administered in combination with inhibitors of “I-kappaB kinase” (IKK inhibitors), as are described for example in WO2001000610, WO2001030774, WO2004022553 or WO2005097129.

In one embodiment, the compound of the formula I is administered in combination with modulators of the glucocorticoid receptor, like those described for example in WO2005090336.

In a further embodiment, the compound of the formula I is administered in combination with CART modulators (see “Cocaine-amphetamine-regulated transcript influences energy metabolism, anxiety and gastric emptying in mice” Asakawa, A. et al.: Hormone and Metabolic Research (2001), 33(9), 554-558);

NPY antagonists such as, for example, naphthalene-1-sulfonic acid {4-[(4-amino-quinazolin-2-ylamino)methyl]cyclohexylmethyl} amide hydrochloride (CGP 71683A);

peptide YY 3-36 (PYY3-36) or analogous compounds, such as, for example, CJC-1682 (PYY3-36 conjugated with human serum albumin via Cys34), CJC-1643 (derivative of PYY3-36 which conjugates in vivo to serum albumin) or those as are described in WO2005080424;

CB1R (cannabinoid receptor 1) antagonists (such as, for example, rimonabant, SR147778, SLV-319, AVE-1625, MK-0364 or salts thereof or those as are described for example in EP 0656354, WO 00/15609, WO2001/64632, WO2001/64633, WO2001/64634, WO02/076949, WO2005080345, WO2005080328, WO2005080343, WO2005075450, WO2005080357, WO200170700, WO2003026647-48, WO200302776, WO2003040107, WO2003007887, WO2003027069, U.S. Pat. No. 6,509,367, WO200132663, WO2003086288, WO2003087037, WO2004048317, WO2004058145, WO2003084930, WO2003084943, WO2004058744, WO2004013120, WO2004029204, WO2004035566, WO2004058249, WO2004058255, WO2004058727, WO2004069838, US20040214837, US20040214855, US20040214856, WO2004096209, WO2004096763, WO2004096794, WO2005000809, WO2004099157, US20040266845, WO2004110453, WO2004108728, WO2004000817, WO2005000820, US20050009870, WO200500974, WO2004111033-34, WO200411038-39, WO2005016286, WO2005007111, WO2005007628, US20050054679, WO2005027837, WO2005028456, WO2005063761-62, WO2005061509, WO2005077897, WO2006047516, WO2006060461, WO2006067428 or WO2006067443);

MC4 agonists (e.g. 1-amino-1,2,3,4-tetrahydronaphthalene-2-carboxylic acid [2-(3a-benzyl-2-methyl-3-oxo-2,3,3,4,6,7-hexahydropyrazolo[4,3-c]pyridin-5-yl)-1-(4-chlorophenyl)-2-oxoethyl]amide; (WO 01/91752)) or LB53280, LB53279, LB53278 or THIQ, MB243, RY764, CHIR-785, PT-141 or those that are described in WO2005060985, WO2005009950, WO2004087159, WO2004078717, WO2004078716, WO2004024720, US20050124652, WO2005051391, WO2004112793, WOUS20050222014, US20050176728, US20050164914, US20050124636, US20050130988, US20040167201, WO2004005324, WO2004037797, WO2005042516, WO2005040109, WO2005030797, US20040224901, WO200501921, WO200509184, WO2005000339, EP1460069, WO2005047253, WO2005047251, EP1538159, WO2004072076 or WO2004072077;

orexin receptor antagonists (e.g. 1-(2-methylbenzoxazol-6-yl)-3-[1,5]naphthyridin-4-ylurea hydrochloride (SB-334867-A) or those as are described for example in WO200196302, WO200185693, WO2004085403 or WO2005075458);
histamine H3 receptor agonists (e.g. 3-cyclohexyl-1-(4,4-dimethyl-1,4,6,7-tetrahydroimidazo[4,5-c]pyridin-5-yl)propan-1-one oxalic acid salt (WO 00/63208) or those as are described in WO200064884 or WO2005082893;
CRF antagonists (e.g. [2-methyl-9-(2,4,6-trimethylphenyl)-9H-1,3,9-triaz afluoren-4-yl]dipropylamine (WO 00/66585));
CRF BP antagonists (e.g. urocortin);
urocortin agonists;
β3 agonists (such as, for example, 1-(4-chloro-3-methanesulfonylmethylphenyl)-2-[2-(2,3-dimethyl-1H-indol-6-yloxy)ethylamino]ethanol hydrochloride (WO 01/83451));
MSH (melanocyte-stimulating hormone) agonists;
MCH (melanin-concentrating hormone) receptor antagonists (such as, for example, NBI-845, A-761, A-665798, A-798, ATC-0175, T-226296, T-71, GW-803430 or compounds such as are described in WO2003/15769, WO2005085200, WO2005019240, WO2004011438, WO2004012648, WO2003015769, WO2004072025, WO2005070898, WO2005070925, WO2004039780, WO2003033476, WO2002006245, WO2002002744, WO2003004027 or FR2868780);
CCK-A agonists (such as, for example, {2-[4-(4-chloro-2,5-dimethoxyphenyl)-5-(2-cyclohexylethyl)thiazol-2-ylcarbamoyl]-5,7-dimethylindol-1-yl}acetic acid trifluoro acetic acid salt (WO 99/15525), SR-146131 (WO 0244150) or SSR-125180);
serotonin reuptake inhibitors (e.g. dexfenfluramine);
mixed serotoninergic and noradrenergic compounds (e.g. WO 00/71549);
5-HT receptor agonists, e.g. 1-(3-ethylbenzofuran-7-yl)piperazine oxalic acid salt (WO 01/09111);
5-HT2C receptor agonists (such as, for example, APD-356, BVT-933 or those as are described in WO200077010, WO20077001-02, WO2005019180, WO2003064423, WO200242304 or WO2005082859);
5-HT6 receptor antagonists as are described for example in WO2005058858;
bombesin receptor agonists (BRS-3 agonists);
galanin receptor antagonists;
growth hormone (e.g. human growth hormone or AOD-9604);
growth hormone-releasing compounds (tertiary butyl 6-benzyloxy-1-(2-diisopropyl-aminoethylcarbamoyl)-3,4-dihydro-1H-isoquinoline-2-carboxylate (WO 01/85695));
growth hormone secretagogue receptor antagonists (ghrelin antagonists) such as, for example, A-778193 or those as are described in WO2005030734;
TRH agonists (see, for example, EP 0 462 884);
uncoupling protein 2 or 3 modulators;
leptin agonists (see, for example, Lee, Daniel W.; Leinung, Matthew C.; Rozhayskaya-Arena, Marina; Grasso, Patricia. Leptin agonists as a potential approach to the treatment of obesity.

### Drugs of the Future (2001), 26(9), 873-881);

DA agonists (bromocriptine or Doprexin);
lipase/amylase inhibitors (e.g. WO 00/40569);
inhibitors of diacylglycerol O-acyltransferases (DGATs) as described for example in US2004/0224997, WO2004094618, WO200058491, WO2005044250, WO2005072740, JP2005206492 or WO2005013907;
inhibitors of fatty acid synthase (FAS) such as, for example, C75 or those as described in WO2004005277;
oxyntomodulin;
oleoyl-estrone;
or thyroid hormone receptor agonists such as, for example: KB-2115 or those as described in WO20058279, WO200172692, WO200194293, WO2003084915, WO2004018421 or WO2005092316.

In one embodiment of the invention, the further active ingredient is leptin;

see, for example, “Perspectives in the therapeutic use of leptin”, Salvador, Javier; Gomez-Ambrosi, Javier; Fruhbeck, Gema, Expert Opinion on Pharmacotherapy (2001), 2(10), 1615-1622.

In one embodiment, the further active ingredient is dexamphetamine or amphetamine.

In one embodiment, the further active ingredient is fenfluramine or dexfenfluramine.

In another embodiment, the further active ingredient is sibutramine.

In one embodiment, the further active ingredient is mazindole or phentermine.

In one embodiment, the compound of the formula I is administered in combination with bulking agents, preferably insoluble bulking agents (see, for example, Carob/Caromax® (Zunft H J; et al., Carob pulp preparation for treatment of hypercholesterolemia, ADVANCES IN THERAPY (2001 September-October), 18(5), 230-6). Caromax is a carob-containing product from Nutrinova, Nutrition Specialties & Food Ingredients GmbH, Industriepark Höchst, 65926 Frankfurt/Main. Combination with Caromax® is possible in one preparation or by separate administration of compounds of the formula I and Caromax®. Caromax® can in this connection also be administered in the form of food products such as, for example, in bakery products or muesli bars.

It will be appreciated that every suitable combination of the compounds of the invention with one or more of the aforementioned compounds and optionally one or more other pharmacologically active substances is regarded as falling within the protection conferred by the present invention.

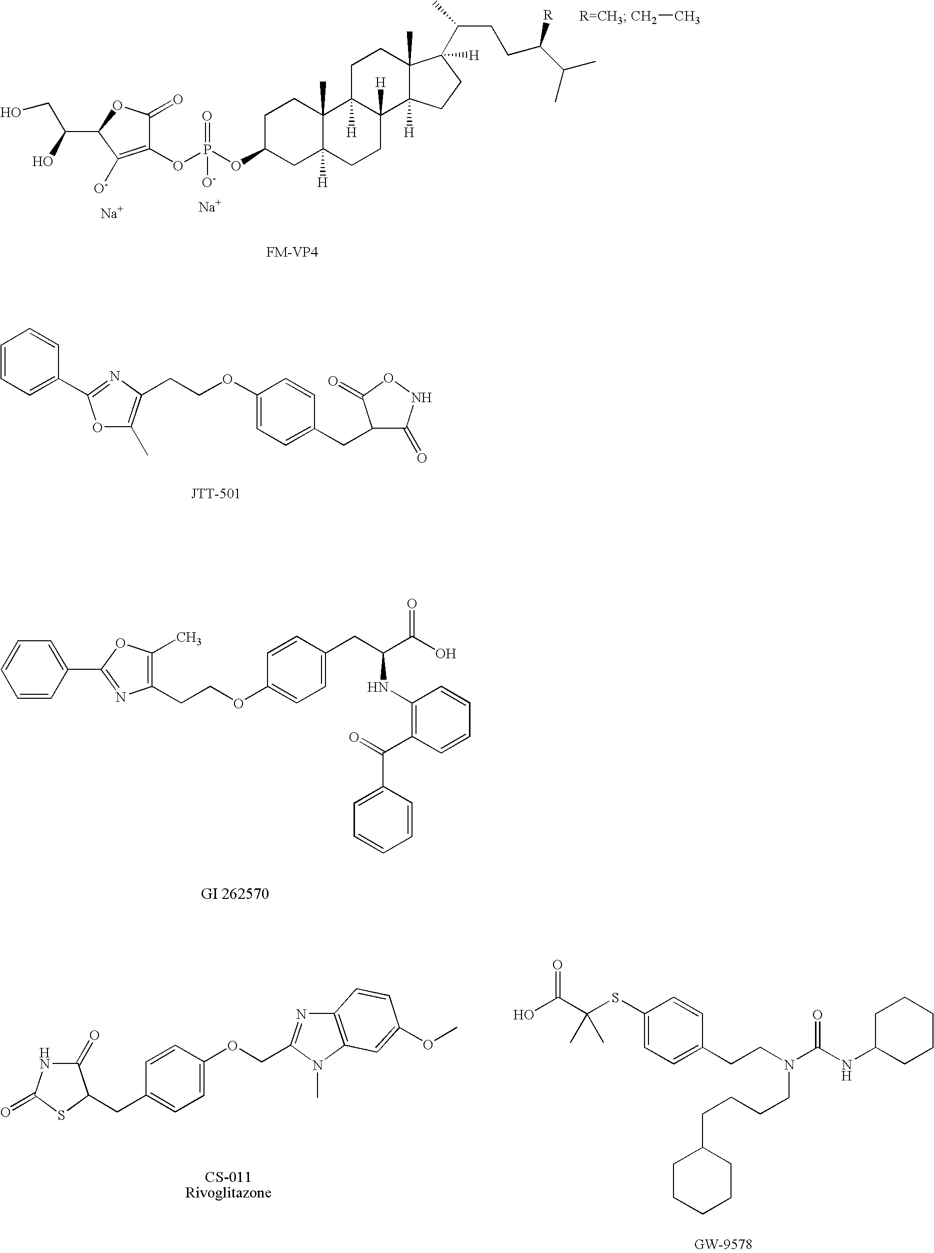

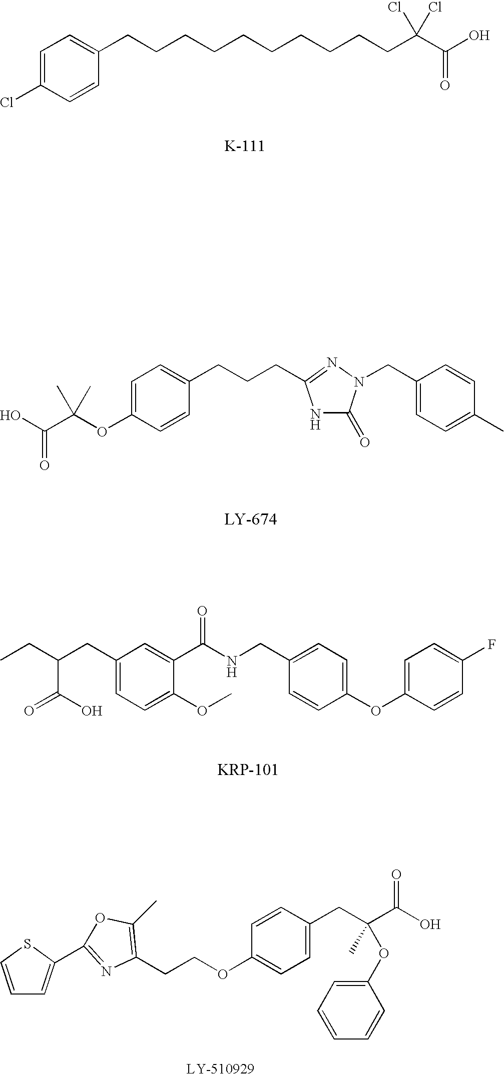

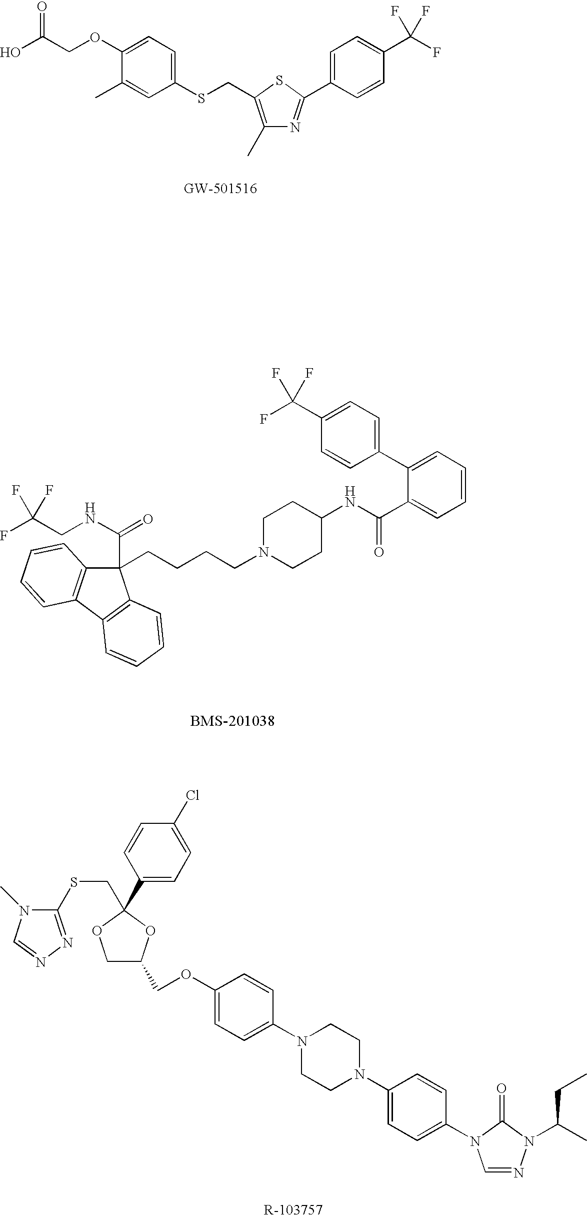

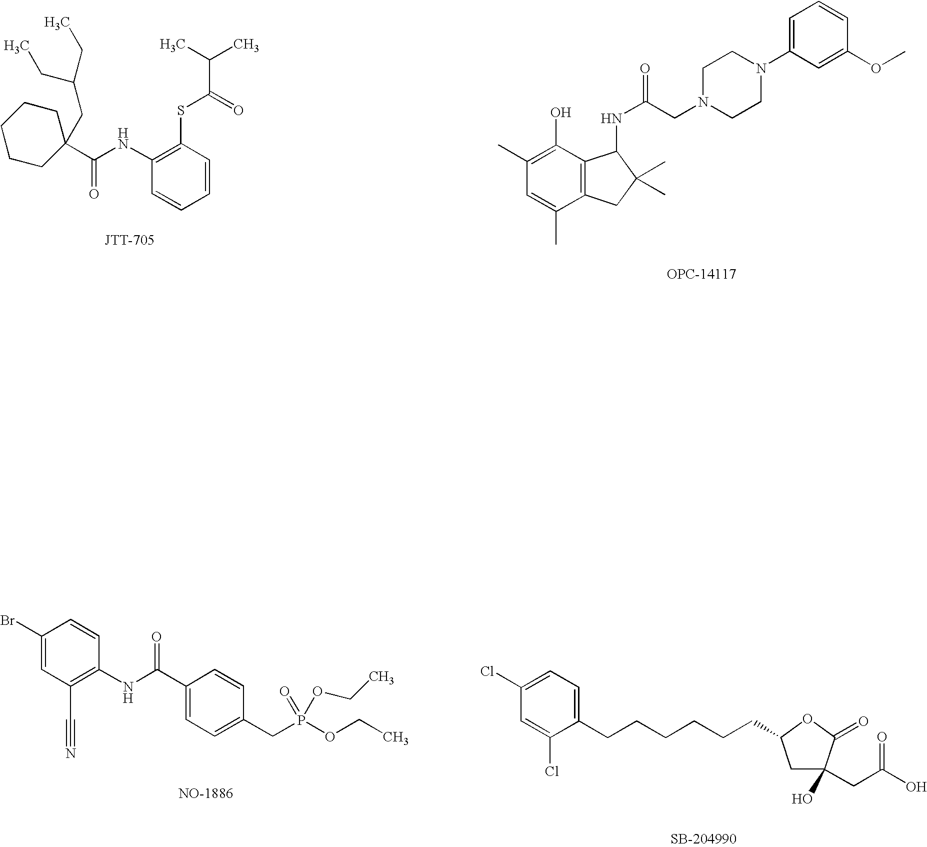

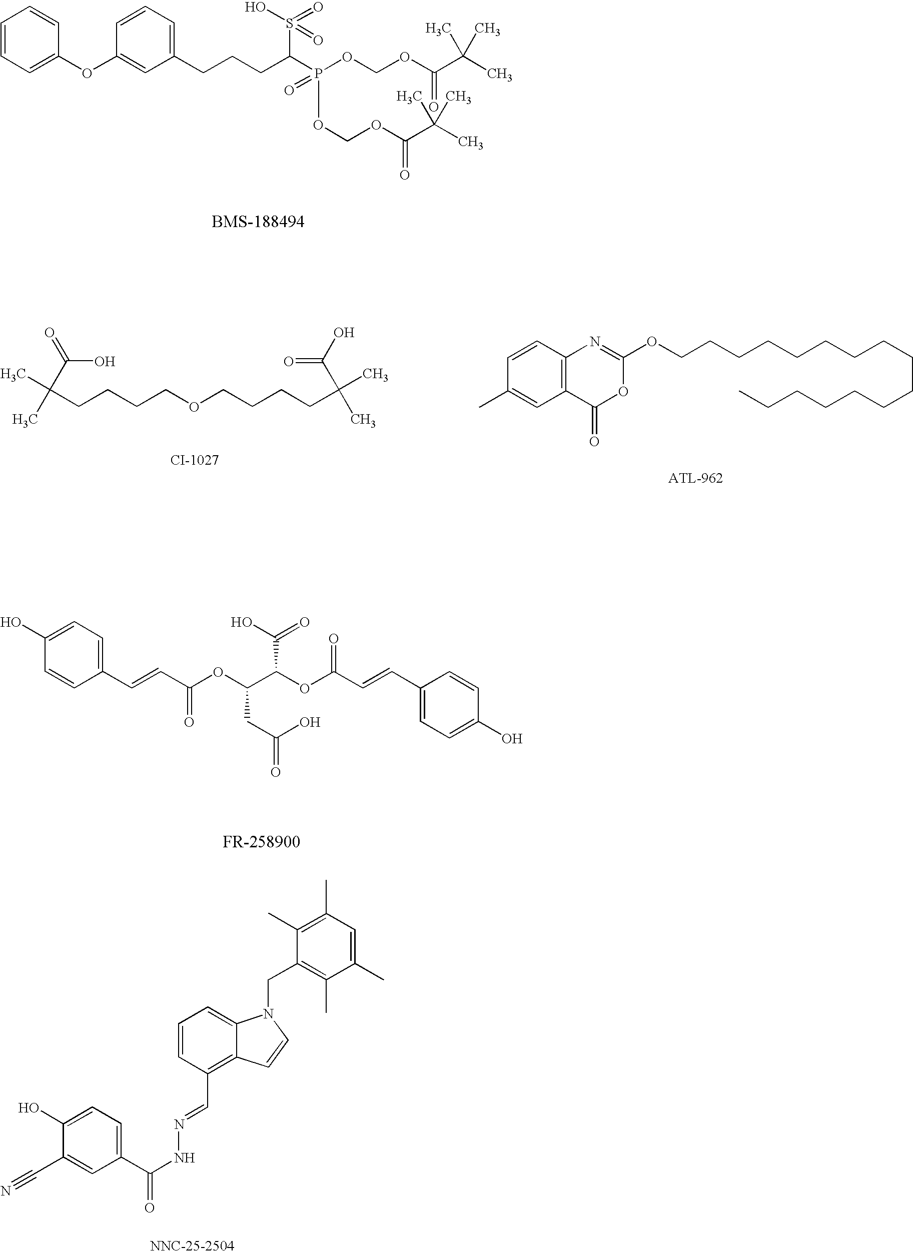

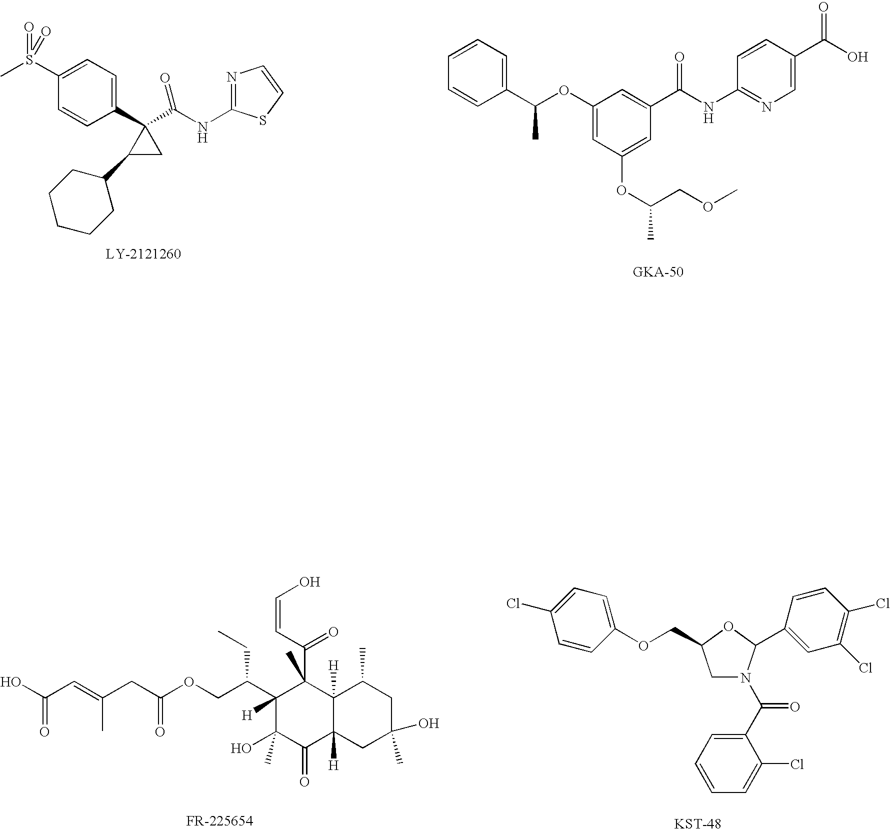

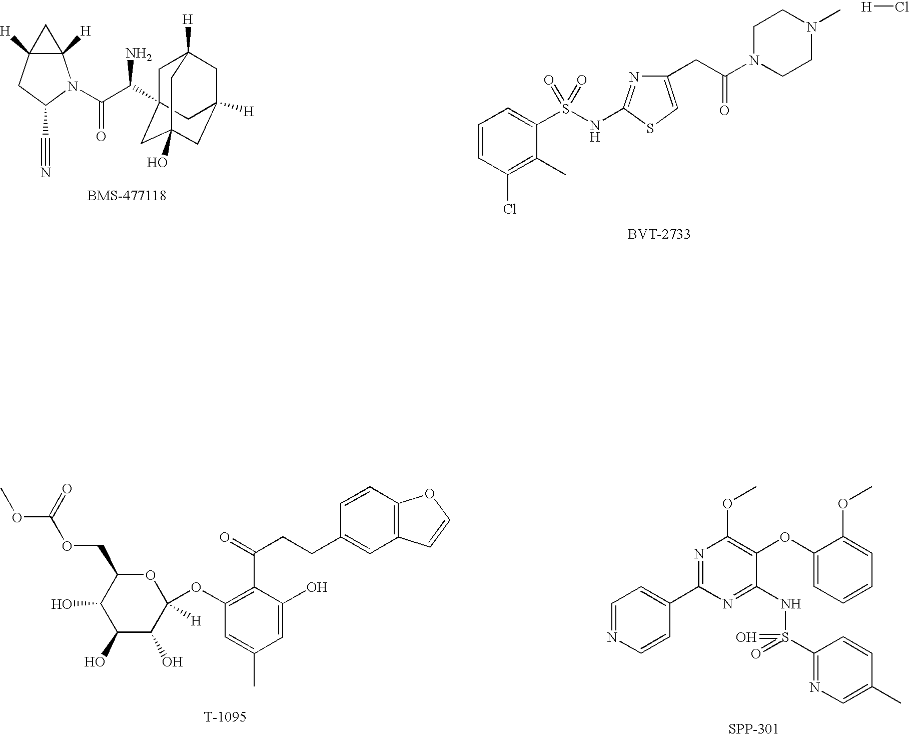

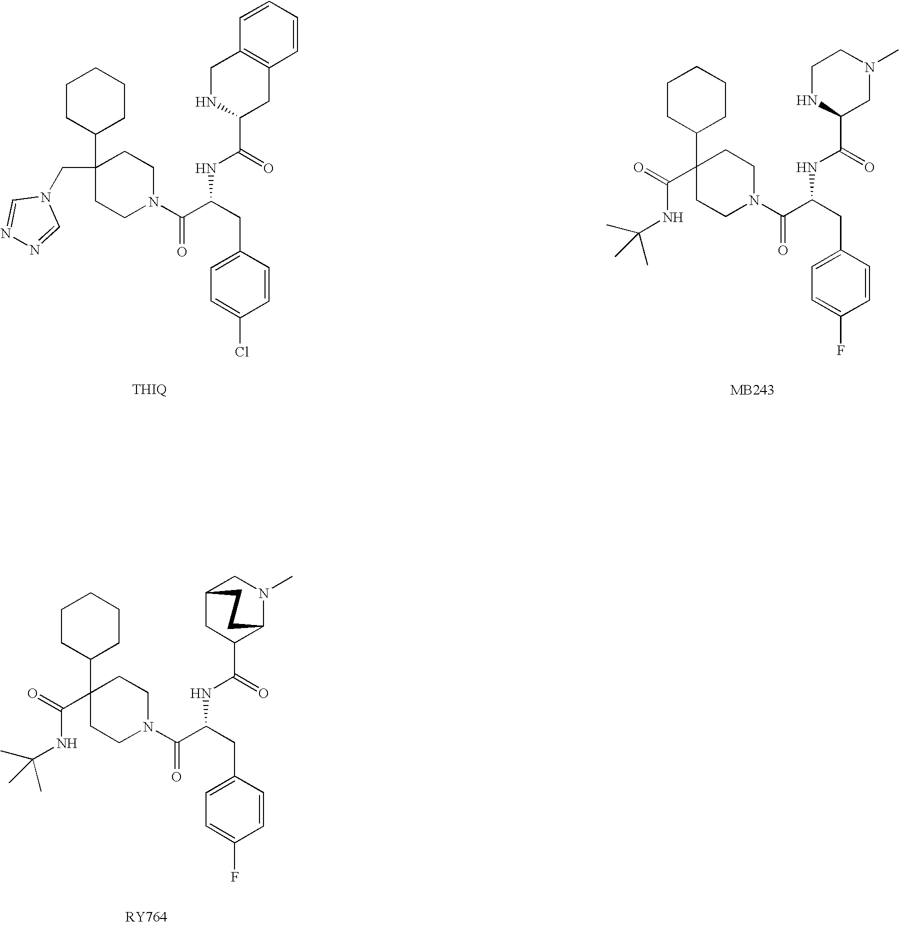

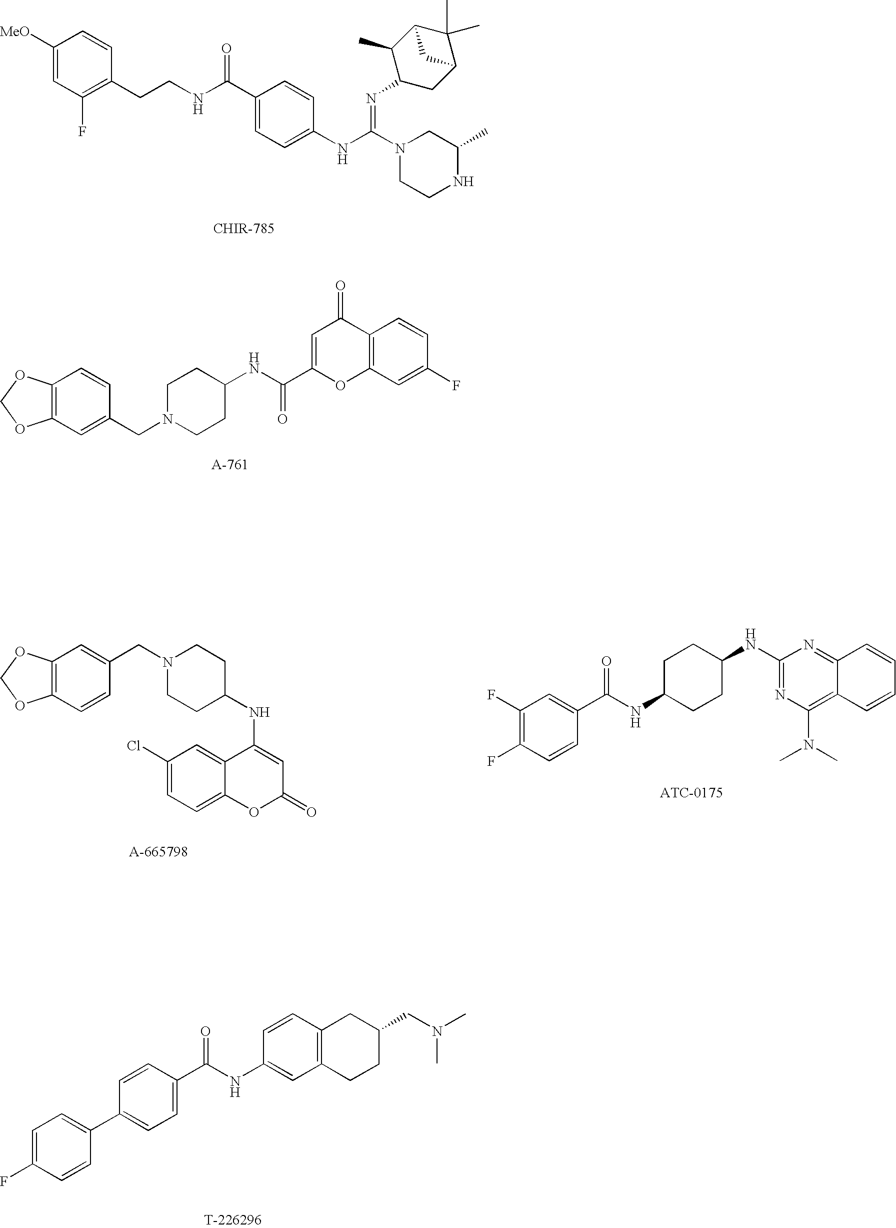

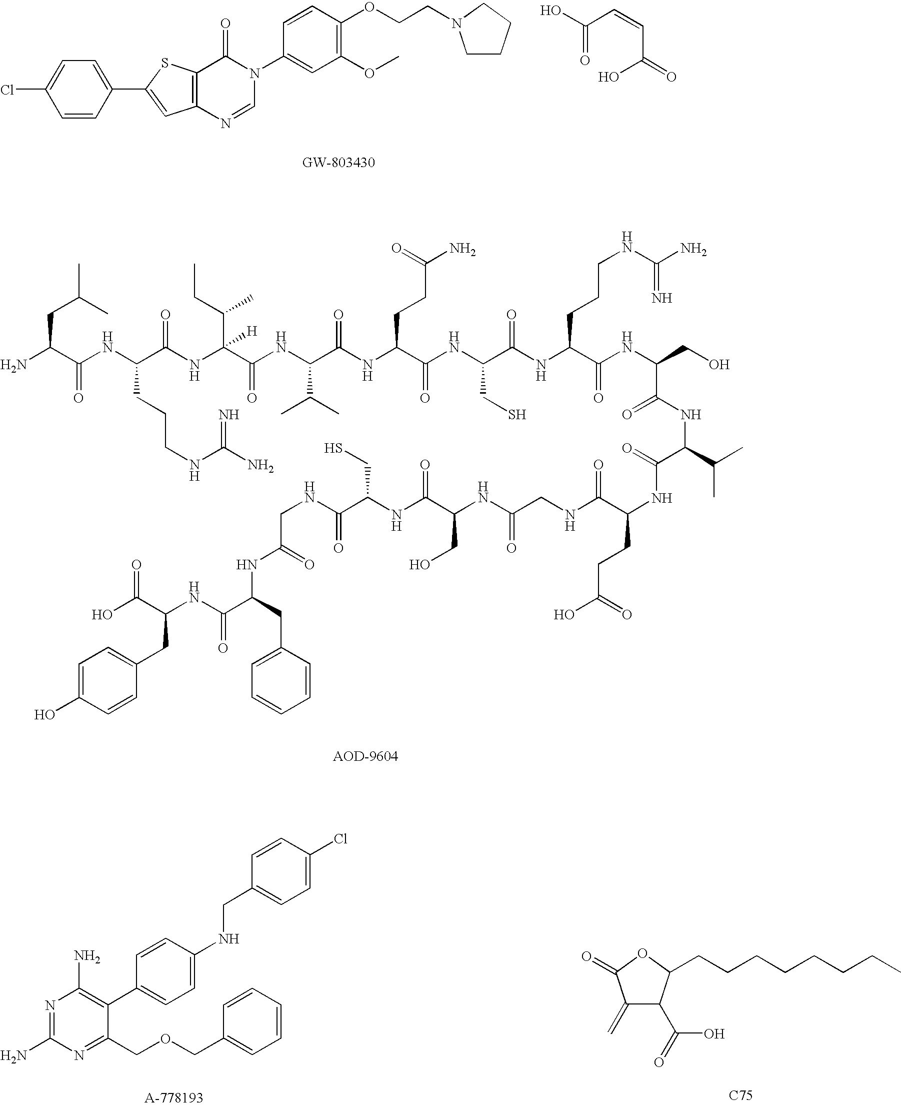

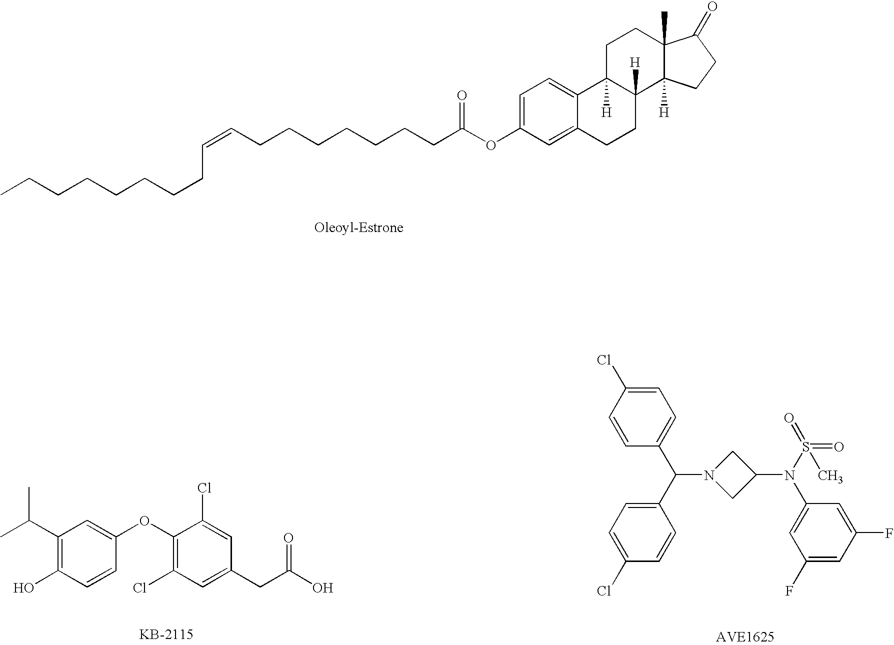

The following examples serve to explain the invention in more detail without restricting the latter to the products and embodiments described in the examples.

TABLE 1
I

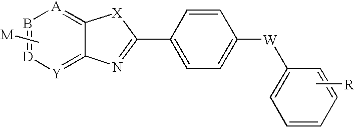

| Ex. | R | W | X | A | B | D | Y | M [bonding site on atom] |
|---|---|---|---|---|---|---|---|---|
| 2 | H | O | NH | CH | CH | CH | CH | CONHCH₂CH₂CH₂-(pyridyl-3-yl); [B] |
| 3 | H | O | NH | CH | CH | CH | CH | CONHCH₂CH₂CH₂-phenyl; [B] |
| 4 | H | O | NH | CH | CH | CH | CH | CONHCH₂CH₂-phenyl; [B] |
| 5 | H | O | NH | CH | CH | CH | CH | CONHCH₂CH₂CH(CH₃)₂; [B] |
| 6 | H | O | NH | CH | CH | CH | CH | CONHCH₂CH₂-cyclopropyl; [B] |
| 7 | H | O | NH | CH | CH | CH | CH | CONHCH₂-cyclopropyl; [B] |
| 8 | H | O | NH | CH | CH | CH | CH | CONHCH₂-(5-chlorothiophen-2-yl); [B] |
| 9 | H | O | NH | CH | CH | CH | CH | CONHCH₂-(thiazol-2-yl); [B] |
| 10 | H | O | NH | CH | CH | CH | CH | CONHCH₂CH₂CH₂—O—CH₂CH₂CH₂CH₃; [B] |
| 12 | 2-Cl | O | NH | CH | CH | CH | CH | CONHCH₂CH₂-phenyl; [B] |
| 13 | 2-Cl | O | NH | CH | CH | CH | CH | CONHCH₂CH₂CH(CH₃)₂; [B] |
| 14 | 2-Cl | O | NH | CH | CH | CH | CH | CONHCH₂CH₂-cyclopropyl; [B] |
| 15 | 2-Cl | O | NH | CH | CH | CH | CH | CONHCH₂-(5-chlorothiophen-2-yl); [B] |
| 16 | 2-Cl | O | NH | CH | CH | CH | CH | CONHCH₂-(thiazol-2-yl); [B] |
| 18 | 2-CF₃ | O | NH | CH | CH | CH | CH | CONHCH₂-(5-chlorothiophen-2-yl); [D] |
| 19 | 2-CF₃ | CO | NH | CH | CH | CH | CH | CONHCH₂-(thiophen-2-yl); [B] |
| 20 | 2-CF₃ | CO | NH | CH | CH | CH | CH | CONHCH₂CH(CH₃)₂; [B] |
| 22 | 2-CF₃ | O | S | CH | CH | CH | CH | CONHCH₂CH(CH₃)₂; [D] |
| 23 | 2-CF₃ | O | NH | CH | CH | CH | CH | CONHCH₂CH₂-cyclopropyl; [D] |
| 24 | 2-CF₃ | O | NH | CH | CH | CH | CH | CONHCH₂CH(CH₃)₂; [D] |
| 25 | 2-CF₃ | O | NH | CH | CH | CH | CH | CONHCH₂-(thiazol-2-yl); [D] |
| 26 | 2-CF₃ | O | NH | CH | CH | CH | CH | CONHCH₂CH₂CH₂-phenyl; [D] |
| 27 | 2-CF₃ | CO | NH | CH | CH | CH | CH | CONHCH₂-(5-chlorothiophen-2-yl); [D] |
| 28 | 2-CF₃ | CO | NH | CH | CH | CH | CH | CONHCH₂-(thiazol-2-yl); [B] |

The activity of the compounds of the invention of the formula I was investigated in the following assay system with microsomal enzymes:

The livers of male Wistar rats which (to induce SCD1 expression) have been fed with a carbohydrate-rich, low-fat diet were homogenized with a Potter tissue homogenizer and differential centrifugation in a buffer which contained 250 mM sucrose and 5 mM HEPES (pH 7.0). The resuspended microsome fraction was stored at −80° C. The stearoyl-CoA desaturase activity was determined in a thin-layer chromatography assay with lit-labeled stearic acid. Stated briefly, the respective compounds of the invention (in DMSO in a final concentration of 1% [v/v]) were incubated with 15 μg of the rat liver microsomes in 200 μl of assay buffer (6 mM MgCl₂, 250 mM sucrose, 150 mM KCl, 40 mM NaF, 100 mM Na₂HPO₄ (pH 7.4), 1.3 mM ATP, 1.5 mM reduced glutathione, 60 μM CoA, 0.33 mM nicotinamide and 0.94 mM NADH) at room temperature for 10 min. 0.5 μCi of [1-¹⁴C]-stearic acid (55 mCi/mmol) was added, and the mixture was incubated at 37° C. for 1 h. The radiolabeled fatty acids were then hydrolyzed with 2.5 M KOH/MeOH:H₂O (4:1) at 65° C. for 4 h, protonated with 280 μl of formic acid and extracted with 500 μl of hexane. The TLC plates were immersed in 10% AgNO₃ and heat-activated before use. 150 μl of the hexane phase were put on the plates, and the TLC plates were developed in buffer (chloroform:methanol:acetic acid:water [90:8:1:0.8]) and dried. The plates were read in a PhosphoImager to quantify the SCD1 activity.

The skilled person is able to modify this assay to determine the inhibition of stearoyl-CoA desaturase activity in various respects. Representative compounds of the invention like those described in the examples showed, when assayed at a concentration of 10 μM in this assay, activity as inhibitors of SCD1, which is indicated as percentage inhibition of SCD1 activity.

TABLE 2
Biological activity of selected compounds

| Ex. | % Inhibition (10 μM) |
|---|---|
| 8 | 37 |
| 9 | 29 |
| 13 | 58 |
| 14 | 80 |
| 15 | 95 |
| 16 | 86 |
| 19 | 100 |
| 20 | 77 |
| 23 | 96 |
| 24 | 77 |
| 25 | 100 |
| 27 | 100 |
| 28 | 100 |

The compounds of the formula I inhibit SCD1 activity and are therefore very suitable for treating lipid metabolism disorders, obesity and the metabolic syndrome (Hulver et al. Cell Metabolism (2005), 2(4), 251-261 and Warensjoe et al. Diabetologia (2005), 48(10), 1999-2005).

Owing to the inhibition of SCD activity, the compounds of the formula I can also be used for the treatment or prevention of further SCD-mediated diseases or of an SCD-mediated condition in a mammal, preferably a human.

The compounds of the present invention are particularly suitable for the treatment and/or prevention of:
1.—obesity, especially visceral (abdominal) obesity
2.—disorders of fatty acid metabolism and glucose utilization disorders
   disorders in which insulin resistance is involved
3. Diabetes mellitus, especially type 2 diabetes, including the prevention of the sequelae associated therewith.
   Particular aspects in this connection are
   hyperglycemia,
   improvement in insulin resistance,
   improvement in glucose tolerance,
   protection of the pancreatic B cells
   prevention of macro- and microvascular disorders
4. Dyslipidemias and their sequelae such as, for example, atherosclerosis, coronary heart disease, cerebrovascular disorders etc., especially those (but not restricted thereto) which are characterized by one or more of the following factors:
   high plasma triglyceride concentrations, high postprandial plasma triglyceride concentrations,
   low HDL cholesterol concentration
   low apoA lipoprotein concentrations
   high LDL cholesterol concentrations
   small dense LDL cholesterol particles
   high apoB lipoprotein concentrations
   desaturation index (e.g. ratio 18:1/18:0n-9, 16:1/16:0 n-7 or 18:1n-9+16:1n-7/16:0 fatty acids)
5. Various other conditions which may be associated with the metabolic syndrome or syndrome X, such as:
   increased abdominal girth
   dyslipidemia (e.g. hypertriglyceridemia and/or low HDL)
   insulin resistance
   hypercoagulability
   hyperuricemia
   microalbuminemia
   thromboses, hypercoagulable and prothrombotic states (arterial and venous)
   high blood pressure
   heart failure such as, for example (but not restricted thereto), following myocardial infarction, hypertensive heart disease or cardiomyopathy
6. Hepatic disorders and conditions related thereto
   fatty liver
   hepatic steatosis
   non-alcoholic hepatitis
   non-alcoholic steatohepatitis (NASH)
   alcoholic hepatitis
   acute fatty liver
   fatty liver of pregnancy
   drug-induced hepatitis
   iron storage diseases
   hepatic fibrosis
   hepatic cirrhosis
   hepatoma
   viral hepatitis
7. Skin disorders and conditions and those associated with polyunsaturated fatty acids
   eczema
   acne
   psoriasis
   keloid scar formation or prevention
   other diseases related to mucous membrane fatty acid composition
8. Primary hypertriglyceridemia or secondary hypertriglyceridemias following
   familial histiocytic reticulosis
   lipoprotein lipase deficiency
   hyperlipoproteinemias
   apolipoprotein deficiency (e.g. apoCII or apoE deficiency)
9. Diseases or conditions related to neoplastic cellular proliferation
   benign or malignant tumors
   cancer
   neoplasias
   metastases
   carcinogenesis
10. Diseases or conditions related to neurological, psychiatric or immune disorders or conditions
11. Other diseases or conditions in which inflammatory reactions, cell differentiation and/or other SCD-mediated aspects may for example be involved are:
   atherosclerosis such as, for example (but not restricted thereto), coronary sclerosis including angina pectoris or myocardial infarction, stroke, ischemic stroke and transient ischemic attack (TIA)
   peripheral occlusive disease
   vascular restenosis or reocclusion
   chronic inflammatory bowel diseases such as, for example, Crohn's disease and ulcerative colitis
   pancreatitis
   sinusitis
   other inflammatory conditions
   retinopathy, ischemic retinopathy
   adipose cell tumors
   lipomatous carcinomas such as, for example, liposarcomas
   solid tumors and neoplasms such as, for example (but not restricted thereto), carcinomas of the gastrointestinal tract, of the liver, of the biliary tract and of the pancreas, endocrine tumors, carcinomas of the lungs, of the kidneys and the urinary tract, of the genital tract, prostate carcinomas etc.
   acute and chronic myeloproliferative disorders and lymphomas
   angiogenesis
   neurodegenerative disorders
   Alzheimer's disease
   multiple sclerosis
   Parkinson's disease
   erythemato-squamous dermatoses such as, for example, psoriasis
   acne vulgaris
   other skin disorders and dermatological conditions which are modulated by PPAR
   eczemas and neurodermatitis
   dermatitis such as, for example, seborrheic dermatitis or photodermatitis
   keratitis and keratoses such as, for example, seborrheic keratoses, senile keratoses, actinic keratoses, photo-induced keratoses or keratosis follicularis
   keloids and keloid prophylaxis
   warts, including condylomata or condylomata acuminata
   human papilloma viral (HPV) infections such as, for example, venereal papillomata, viral warts such as, for example, molluscum contagiosum, leukoplakia
   papular dermatoses such as, for example, lichen planus
   skin cancer such as, for example, basal-cell carcinomas, melanomas or cutaneous T-cell lymphomas
   localized benign epidermal tumors such as, for example, keratoderma, epidermal naevi
   chilblains
   high blood pressure
   syndrome X
   polycystic ovary syndrome (PCOS)
   asthma
   cystic fibrosis
   osteoarthritis
   lupus erythematosus (LE) or inflammatory rheumatic disorders such as, for example, rheumatoid arthritis
   vasculitis
   wasting (cachexia)
   gout
   ischemia/reperfusion syndrome
   acute respiratory distress syndrome (ARDS)
   viral diseases and infections
   lipodystrophy and lipodystrophic conditions, also for treating adverse drug effects (e.g. after taking medicaments for treating HIV or tumors)
   myopathies and lipid myopathies (such as carnitine palmitoyltransferase I or II deficiency)

Formation of muscles and a lean body or muscle mass formation in animal management and in humans.

### Preparation

The compounds of the invention of the general formula I are prepared by processes known per se in the literature (e.g. McClure, Kelly J.; Huang, Liming; Arienti, Kristen L.; Axe, Frank U.; Brunmark, Anders; Blevitt, Jon; Guy Breitenbucher, J.; Bioorganic & Medicinal Chemistry Letters (2006), 16(7), 1924-1928; Arienti, Kristen L.; Brunmark, Anders; Axe, Frank U.; McClure, Kelly; Lee, Alice; Blevitt, Jon; Neff, Danielle K.; Huang, Liming; Crawford, Shelby; Pandit, Chemagiri R.; Karlsson, Lars; Breitenbucher, J. Guy, Journal of Medicinal Chemistry (2005), 48(6), 1873-1885) and can be obtained in accordance with the following reaction sequences, in which the radicals have the meanings indicated above.

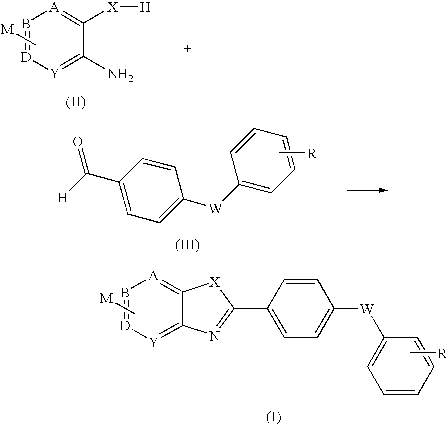

The compounds used as starting materials are commercially available or can be prepared by processes known from the literature; 4-phenoxy-substituted benzaldehydes for example can be obtained by reacting appropriately substituted phenols with 4-fluoro- or 4-chlorobenzaldehyde in the presence of a base (e.g. Pfefferkorn, Jeffrey A.; Greene, Meredith L.; Nugent, Richard A.; Gross, Rebecca J.; Mitchell, Mark A.; Finzel, Barry C.; Harris, Melissa S.; Wells, Peter A.; Shelly, John A.; Anstadt, Robert A.; Kilkuskie, Robert E.; Kopta, Laurice A.; Schwende, Francis J.; Bioorganic & Medicinal Chemistry Letters (2005), 15(10), 2481-2486).

On the other hand, these compounds can also be prepared by reacting 4-bromophenol with haloaromatic compounds and subsequent conversion into the corresponding aldehydes.

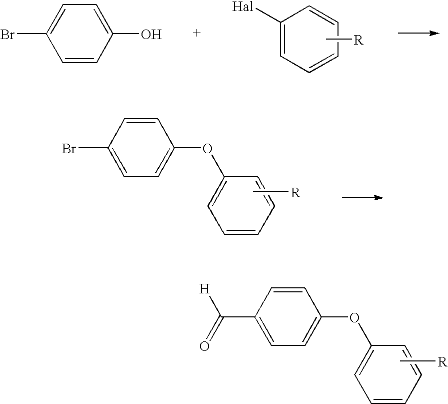

Diaminopyridine carboxylic esters can be prepared according to P. Chand et. al., Bioorganic & Medicinal Chemistry Letters (2005), 13(17), 2665-2678. Substituted 4-benzylbenzaldehydes can be prepared in analogy to Langle, Sandrine; Abarbri, Mohamed; Duchene, Alain, Tetrahedron Letters (2003), 44(52), 9255-9258.

Benzoyl-substituted benzaldehydes can be prepared by oxidizing the corresponding alcohols. This method is described in Kashiwagi, Yoshitomo; Ikezoe, Hiroshi; Ono, Tetsuya. Synlett (2006), (1), 69-72; Smith, Amos B., III; Rucker, Paul V.; Brouard, Ignacio; Freeze, B. Scott; Xia, Shujun; Horwitz, Susan Band. Organic Letters (2005), 7(23), 5199-5202.

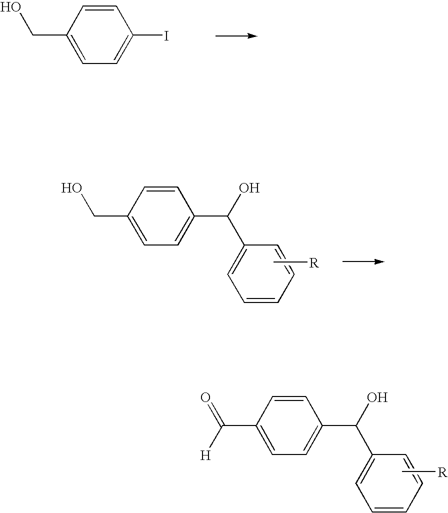

An alternative method uses Friedel-Crafts acylation and subsequent side-chain halogenation and hydrolysis: Nakatani, Kazuhiko; Dohno, Chikara; Saito, Isao. Journal of Organic Chemistry (1999), 64(18), 6901-6904.

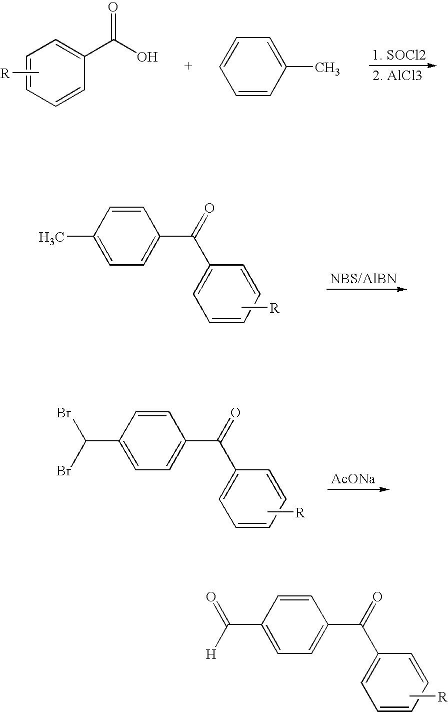

If acids are liberated during these reactions, it is advantageous to add bases such as pyridine, triethylamine, sodium hydroxide solution or alkali metal carbonates to increase the rate. The reactions can be carried out in wide temperature ranges. It has proved advantageous to operate at temperatures from 0° C. to the boiling point of the solvent used. Examples of solvents used are methylene chloride, THF, DMF, toluene, ethyl acetate, n-heptane, dioxane, diethyl ether or pyridine. Under anhydrous conditions, strong bases such as lithium hydride, sodium hydride or potassium tert-butanolate in aprotic solvents such as THF or DMF have also proved suitable.

The compounds of the general formula I are isolated from the reaction mixture and purified by processes known per se, such as extraction, crystallization or chromatography.

The examples detailed below serve to illustrate the invention without, however, restricting it. The identity of the compounds was checked by mass spectrometry.

### EXAMPLE 1

### Ethyl 2-(4-phenoxyphenyl)-3H-benzimidazole-5-carboxylate

Sodium metabisulfite (10.55 g, 55.49 mmol) in 50 ml of water was added dropwise to a solution of 4-phenoxybenzaldehyde (11.0 g, 55.49 mmol) in 250 ml of ethanol under argon. The thick precipitate which separated out was diluted with 50 ml of ethanol, stirred at room temperature for 4 h and stored in a refrigerator overnight. The precipitate was filtered off with suction, washed with ethanol and dried. The isolated intermediate was suspended in 100 ml of DMF and, after addition of ethyl 3,4-diaminobenzoate (10.0 g, 55.49 mmol), stirred at 130° C. for 4 h. DMF was distilled off in vacuo, the residue was mixed with water and ethyl acetate, and the organic phase was separated off, washed with water, dried and concentrated. The resulting crude product (18.3 g) was reacted further.

### EXAMPLE 2

### 2-(4-Phenoxyphenyl)-3H-benzimidazole-5-(3-pyridin-3-ylpropyl)carboxamide

### 2a: 2-(4-Phenoxyphenyl)-3H-benzimidazole-5-carboxylic acid

Ethyl 2-(4-phenoxyphenyl)-3H-benzimidazole-5-carboxylate (2.0 g, 5.58 mmol) was dissolved in 10 ml of THF. Addition of lithium hydroxide hydrate (1.64 g, 39.08 mmol) in 30 ml of water was followed by stirring at room temperature for a total of 22 h. THF was distilled off, and the aqueous phase was extracted with ethyl acetate. The aqueous phase was then acidified with 4N hydrochloric acid, and the precipitated product was filtered off with suction, washed with water and dried. Yield: 1.64 g (89%), M+H+: 331.09.

### 2b: 2-(4-Phenoxyphenyl)-3H-benzimidazole-5-(3-pyridin-3-ylpropyl)carboxamide

Triethylamine (0.15 ml, 1.078 mmol) and HATU (0.246 mg, 0.647 mmol) were added to 2-(4-phenoxyphenyl)-3H-benzimidazole-5-carboxylic acid (178 mg, 0.539 mmol) and 3-pyridin-3-ylpropylamine (80.09 mg, 0.594 mmol) in 10 ml of DMF and stirred at room temperature for 6 h. The reaction mixture was concentrated in vacuo, the residue was mixed with water and ethyl acetate, and the organic phase was separated off, washed with water, dried and concentrated. The resulting crude product was purified by preparative HPLC (RP18, acetonitrile/water 0.1% TFA). Yield: 217 mg (90%), M+H+: 449.29.

### EXAMPLE 3

### 2-(4-Phenoxyphenyl)-3H-benzimidazole-5-(3-phenylpropyl)carboxamide

2-(4-Phenoxyphenyl)-3H-benzimidazole-5-carboxylic acid and 3-phenylpropylamine were reacted in analogy to Example 2b. Yield: 97 mg (40%), M+H+: 448.28.

### EXAMPLE 4

### 2-(4-Phenoxyphenyl)-3H-benzimidazole-5-phenethylcarboxamide

2-(4-Phenoxyphenyl)-3H-benzimidazole-5-carboxylic acid and phenethylamine were reacted in analogy to Example 2b. Yield: 70 mg (30%), M+H+: 434.26.

### EXAMPLE 5

### 2-(4-Phenoxyphenyl)-3H-benzimidazole-5-(3-methylbutyl)carboxamide

2-(4-Phenoxyphenyl)-3H-benzimidazole-5-carboxylic acid and 3-methylbutylamine were reacted in analogy to Example 2b. Yield: 58 mg (27%), M+H+: 400.27.

### EXAMPLE 6

### 2-(4-Phenoxyphenyl)-3H-benzimidazole-5-(2-cyclopropylethyl)carboxamide

2-(4-Phenoxyphenyl)-3H-benzimidazole-5-carboxylic acid and 2-cyclopropylethylamine hydrochloride were reacted in analogy to Example 2b. Yield: 26 mg (12%), M+H+: 398.16.

### EXAMPLE 7

### 2-(4-Phenoxyphenyl)-3H-benzimidazole-5-cyclopropylmethylcarboxamide

2-(4-Phenoxyphenyl)-3H-benzimidazole-5-carboxylic acid and C-cyclopropylmethylamine were reacted in analogy to Example 2b. Yield: 74 mg (36%), M+H+: 384.14.

### EXAMPLE 8

### 2-(4-Phenoxyphenyl)-3H-benzimidazole-5-(5-chlorothiophen-2-ylmethyl)carboxamide

2-(4-Phenoxyphenyl)-3H-benzimidazole-5-carboxylic acid and C-(5-chlorothiophen-2-yl)methylamine were reacted in analogy to Example 2b. Yield: 65 mg (26%), M+H+: 460.06.

### EXAMPLE 9

### 2-(4-Phenoxyphenyl)-3H-benzimidazole-5-(thiazol-2-ylmethyl)carboxamide

2-(4-Phenoxyphenyl)-3H-benzimidazole-5-carboxylic acid and C-(thiazol-2-yl)methylamine were reacted in analogy to Example 2b. Yield: 84 mg (37%), M+H+: 427.09.

### EXAMPLE 10

### 2-(4-Phenoxyphenyl)-3H-benzimidazole-5-(3-butoxypropyl)carboxamide

2-(4-Phenoxyphenyl)-3H-benzimidazole-5-carboxylic acid and 3-butoxypropylamine were reacted in analogy to Example 2b. Yield: 111 mg (46%), M+H+: 444.17.

### EXAMPLE 11

### Ethyl 2-[4-(2-chlorophenoxy)phenyl]-3H-benzimidazole-5-carboxylate

4-(2-Chlorophenoxy)benzaldehyde and ethyl 3,4-diaminobenzoate were reacted in analogy to Example 1. Yield: 1.75 g (100%), M+H+: 393.17.

### EXAMPLE 12

### 2-[4-(2-Chlorophenoxy)phenyl]-3H-benzoimidazole-5-phenethylcarboxamide

### 12a: 2-[4-(2-Chlorophenoxy)phenyl]-3H-benzimidazole-5-carboxylic acid

Ethyl 2-[4-(2-chlorophenoxy)phenyl]-3H-benzimidazole-5-carboxylate was reacted in analogy to Example 2a. Yield: 1.49 g (100%), M+H+: 365.08.

### 12b: 2-[4-(2-Chlorophenoxy)phenyl]-3H-benzoimidazole-5-phenethylcarboxamide

2-[4-(2-Chlorophenoxy)phenyl]-3H-benzimidazole-5-carboxylic acid and phenethylamine were reacted in analogy to Example 2b. Yield: 65 mg (34%), M+H+: 468.36.

### EXAMPLE 13

### 2-[4-(2-Chlorophenoxy)phenyl]-3H-benzoimidazole-5-(3-methylbutyl)carboxamide

2-[4-(2-Chlorophenoxy)phenyl]-3H-benzimidazole-5-carboxylic acid and 3-methylbutylamine were reacted in analogy to Example 2b. Yield: 117 mg (66%), M+H+: 434.35.

### EXAMPLE 14

### 2-[4-(2-Chlorophenoxy)phenyl]-3H-benzoimidazole-5-(2-cyclopropylethyl)carboxamide

2-[4-(2-Chlorophenoxy)phenyl]-3H-benzimidazole-5-carboxylic acid and 2-cyclopropylethylamine hydrochloride were reacted in analogy to Example 2b. Yield: 79 mg (45%), M+H+: 432.33.

### EXAMPLE 15

### 2-[4-(2-Chlorophenoxy)phenyl]-3H-benzoimidazole-5-(5-chlorothiophen-2-yl-methyl)carboxamide

2-[4-(2-Chlorophenoxy)phenyl]-3H-benzimidazole-5-carboxylic acid and C-(5-chlorothiophen-2-yl)methylamine were reacted in analogy to Example 2b. Yield: 95 mg (47%), M+H+: 494.28.

### EXAMPLE 16

### 2-[4-(2-Chlorophenoxy)phenyl]-3H-benzoimidazole-5-(thiazol-2-ylmethyl)carboxamide

2-[4-(2-Chlorophenoxy)phenyl]-3H-benzimidazole-5-carboxylic acid and C-(thiazol-2-yl)methylamine were reacted in analogy to Example 2b. Yield: 99 mg (52%), M+H+: 461.29.

### EXAMPLE 17

### Ethyl 2-[4-(2-trifluoromethylphenoxy)phenyl]-3H-benzimidazole-5-carboxylate

### 17a: 4-(2-Trifluoromethylphenoxy)benzaldehyde

4-Fluorobenzaldehyde (397 mg, 3.2 mmol), 2-hydroxybenzotrifluoride (1.14 g, 7.04 mmol) and cesium carbonate (1.04 g, 7.04 mmol) were stirred in 20 ml of DMF at 90° C. for 6 h. The reaction mixture was concentrated in vacuo, and the residue was taken up in water and ethyl acetate, and the organic phase was separated off, washed with water, dried and concentrated. The resulting crude product was purified by preparative HPLC(RP18, acetonitrile/water 0.1% TFA). Yield: 108 mg (13%), M+H+: 267.15.

### 17b: Ethyl 2-[4-(2-trifluoromethylphenoxy)phenyl]-3H-benzimidazole-5-carboxylate

Sodium metabisulfite (71.5 mg, 0.376 mmol) in 5 ml of water was added dropwise to a solution of 4-(2-trifluoromethylphenoxy)benzaldehyde (100 mg, 0.376 mmol) in 10 ml of ethanol under argon. The reaction mixture was stirred at room temperature for 4 h and stored in a refrigerator overnight. The reaction mixture was concentrated in vacuo, and the residue was dissolved in 15 ml of DMF and, after addition of ethyl 3,4-diaminobenzoate (67.8 mg, 0.376 mmol), stirred at 130° C. for 6 h. DMF was distilled out in vacuo, the residue was mixed with water and ethyl acetate, and the organic phase was separated off, washed with water, dried and concentrated. The resulting crude product was purified by preparative HPLC (RP18, acetonitrile/water 0.1% TFA). Yield: 24 mg (14%), M+H+: 427.08.

### EXAMPLE 18

### 2-[4-(2-Trifluoromethylphenoxy)phenyl]-3H-benzimidazole-5-(5-chlorothiophen-2-ylmethyl)carboxamide

### 18a: 4-(2-Trifluoromethylphenoxy)bromobenzene

Potassium hydroxide (5.06 g, 90 mmol) was dissolved in 58 ml of a 17/1 DMSO/H2O mixture. 4-Bromophenol (13.5 g, 78 mmol) was added in portions thereto, and the mixture was heated with stirring at 80° C. for 5 minutes. Then 1-fluoro-2-trifluoromethylbenzene (10 g, 61 mmol) was rapidly added dropwise, and the mixture was stirred at 120° C. for 2 h. After cooling to room temperature, 100 ml of water were added. The aqueous phase was extracted 3 times with 150 ml of diethyl ether each time, and the organic phases were combined, washed with water and saturated sodium chloride solution, dried over magnesium sulfate and concentrated. Yield: 13.4 g (69%), M+H+: 317.2.

### 18b: 4-(2-Trifluoromethylphenoxy)benzaldehyde

Under an inert gas atmosphere, 4-(2-trifluoromethylphenoxy)bromobenzene (1.0 g, 3.15 mmol) was dissolved in 20 ml of anhydrous THF and cooled to −78° C. While stirring, 6.31 ml of a butyllithium solution (2N in hexane) were added dropwise. After 5 minutes, 1.46 ml of DMF (18.9 mmol) were added, and the solution was allowed to warm to room temperature while stirring. Addition of 10 ml of ammonium chloride was followed by extraction with dichloromethane, and the organic phases were combined, washed, dried over magnesium sulfate and concentrated. The residue was purified by chromatography (silica gel, mobile phase heptane/ethyl acetate 90:10). Yield: 340 mg (15%), M+H+: 267.1.

### 18c: 2-[4-(2-Trifluoromethylphenoxy)phenyl]-3H-benzimidazole-5-carboxylic acid

3,4-Diaminobenzoic acid (0.465 g, 3.06 mmol) and sodium metabisulfite (0.67 g, 3.612 mmol) were added to a solution of 4-(2-trifluoromethylphenoxy)benzaldehyde (0.74 g, 2.78 mmol) in 3 ml of DMF. The mixture was stirred at 100° C. in a Biotage® microwave reactor for 5 min, cooled and diluted with 20 ml of ethyl acetate, and the organic phase was washed 5 times with 10 ml of water each time, dried over magnesium sulfate and concentrated. Yield: 780 mg (70%), M+H+: 399.0.

### 18d: 2-[4-(2-Trifluoromethylphenoxy)phenyl]-3H-benzimidazole-5-(5-chlorothiophen-2-ylmethyl)carboxamide

1,3-Diethylcarbodiimide (172.5 mg, 0.9 mmol), HOBT (172.5 mg, 0.9 mmol), triethylamine (0.25 ml) and (5-chlorothiophen-2-ylmethyl)amine (82.8 mg, 0.45 mmol) were added to 2-[4-(2-trifluoromethylphenoxy)phenyl]-3H-benzimidazole-5-carboxylic acid (0.1793 g, 0.45 mmol) in 2 ml of acetonitrile. The reaction mixture was stirred at room temperature overnight, mixed with 20 ml of dichloromethane and washed with 10 ml of water, and the organic phase was dried over magnesium sulfate and concentrated. The residue was purified by chromatography (silica gel, mobile phase gradient heptane/ethyl acetate 94:6 to 40:60). Yield: 87 mg (37%), M+H+: 528.0.

### EXAMPLE 19

### 2-[4-(2-Trifluoromethylbenzoyl)phenyl]-3H-benzimidazole-5-(thiophen-2-ylmethyl)-carboxamide

### 19a: 4-(2-Trifluoromethylbenzoyl)benzaldehyde

4.43 ml of a 4M hydrochloric acid solution were added to (4-[1,3]dioxan-2-ylphenyl)-(2-trifluoromethylphenyl)methanone (2.0 g, 5.95 mmol) in 13 ml of a dioxane/water mixture, and the mixture was stirred at room temperature for 60 h. Concentration of the solvents in vacuo was followed by extraction with dichloromethane, and the organic phase was concentrated and chromatographed on silica gel with dichloromethane as mobile phase. Yield: 1.31 g (79%), M+H+: 279.3.

### 19b: 3,4-Dinitro-N-thiophen-2-ylmethylbenzamide

Oxalyl chloride (2.27 ml, 26.4 mmol) and one drop of DMF were added to 3,4-dinitrobenzoic acid (1.0 g, 4.71 mmol) in 11 ml of methylene chloride at −25° C. The reaction mixture was stirred at room temperature overnight and concentrated in vacuo. The residue was dissolved in 11 ml of dichloromethane and, at −5° C., thiophen-2-ylmethylamine (0.56 g, 4.95 mmol) in pyridine (1 ml) and dichloromethane (10 ml) was added dropwise. The reaction mixture was stirred at room temperature for 4 h and concentrated in vacuo. The crude product was chromatographed on silica gel with dichloromethane/methanol 98:2 as mobile phase. Yield: 0.996 g (69%), M+H+: 308.0338.

### 19c: 3,4-Diamino-N-thiophen-2-ylmethylbenzamide

3,4-Dinitro-N-thiophen-2-ylmethylbenzamide (0.41 g, 1.33 mmol) was hydrogenated in 10 ml of ethanol/THF (1/1) in the presence of 40 mg of 10% Pd/C at room temperature for 2 h, filtered through Celite and concentrated. Yield: 320 mg (97%), M+H+: 248.2.

### 19d: 2-[4-(2-Trifluoromethylbenzoyl)phenyl]-3H-benzimidazole-5-(thiophen-2-ylmethyl)-carboxamide

3,4-Diamino-N-thiophen-2-ylmethylbenzamide (100 mg, 0.4 mmol) and 4-(2-trifluoromethyl-benzoyl)benzaldehyde (110 mg, 0.4 mmol) were stirred in 8 ml of dioxane with diacetoxyiodobenzene (191 mg, 0.59 mmol) at room temperature for 2 h. Then diacetoxyiodobenzene (191 mg, 0.59 mmol) was again added, and the mixture was stirred at room temperature overnight and concentrated. The residue was purified by chromatography (silica gel, mobile phase ethyl acetate/cyclohexane 1:6). Yield: 168 mg (84%), M+H+: 506.2.

### EXAMPLE 20

### 2-[4-(2-Trifluoromethylbenzoyl)phenyl]-3H-benzoimidazole-5-isobutylcarboxamide

The compound was prepared in analogy to the process described above.

### EXAMPLE 23

### 2-[4-(2-Trifluoromethylphenoxy)phenyl]-3H-benzimidazole-5-(2-cyclopropylethyl)-carboxamide

The compound was obtained by reaction with 2-cyclopropylethylamine in analogy to Example 18d. Yield: 14%, M+H+: 466.

### EXAMPLE 24

### 2-[4-(2-Trifluoromethylphenoxy)phenyl]-3H-benzimidazole-5-(3-methylbutyl)carboxamide

The compound was obtained by reaction with 3-methylbutylamine in analogy to Example 18d. Yield: 25%, M+H+: 468.

### EXAMPLE 25

### 2-[4-(2-Trifluoromethylphenoxy)phenyl]-3H-benzimidazole-5-(thiazol-2-ylmethyl)carboxamide

The compound was obtained by reaction with thiazol-2-ylmethylamine in analogy to Example 18d. Yield: 40%, M+H+: 495.

### EXAMPLE 26

### 2-[4-(2-Trifluoromethylphenoxy)phenyl]-3H-benzimidazole-5-(3-phenylpropyl)carboxamide

The compound was obtained by reaction with 3-phenylpropylamine in analogy to Example 18d. Yield: 22%, M+H+: 516.

### EXAMPLE 27

### 2-[4-(2-Trifluoromethylbenzoyl)phenyl]-3H-benzimidazole-5-(5-chlorothiophen-2-ylmethyl)-carboxamide

### EXAMPLE 27a

### 4-(2-Trifluoromethylbenzoyl)benzaldehyde

(4-[1,3]Dioxan-2-ylphenyl)-(2-trifluoromethylphenyl)methanone (10.0 g, 29.7 mmol) was stirred in a mixture of 50 ml of 80:20 acetic acid:water at 65° C. for 5.5 h. After cooling to RT, the reaction mixture was poured into an ice-cold saturated sodium bicarbonate solution and then extracted with diethyl ether. The organic phase was washed with water and saturated brine, dried over anhydrous magnesium sulfate and concentrated in vacuo. The residue was purified by column chromatography with a heptane:dichloromethane gradient from 95:5 to 50:50 as mobile phase. Removal of the solvent resulted in 6.51 g of 4-(2-trifluoromethyl-benzoyl)benzaldehyde.

### EXAMPLE 27b

### 2-[4-(2-Trifluoromethylbenzoyl)phenyl]-1H-benzimidazole-5-carboxylic acid

3,4-Diaminobenzoic acid (1.8 g, 11.86 mmol) and Na₂S₂O₅ (2.66 g, 14.02 mmol) were added to a stirred solution of 4-(2-trifluoromethylbenzoyl)benzaldehyde (3.0 g, 10.78 mmol) in 18 ml of DMF. The reaction mixture was heated to 100° C. in a microwave reactor for 30 minutes 3 times and then filtered. The filtrate was concentrated in vacuo, and the oily residue was purified by flash chromatography with a CH₂Cl₂:MeOH:AcOH gradient from 99.2:0.8:0.08 to 92:8:0.8. Concentration resulted in 3.49 g of 2-[4-(2-trifluoromethylbenzoyl)phenyl]-1H-benzimidazole-5-carboxylic acid. M+H+: 411.

### EXAMPLE 27c

The compound was obtained by reacting 2-[4-(2-trifluoromethylbenzoyl)phenyl]-1H-benzimidazole-5-carboxylic acid with 5-chlorothiophen-2-ylmethylamine in analogy to Example 18d. Yield: 41%, M+H+: 540.

### EXAMPLE 28

### 2-[4-(2-Trifluoromethylbenzoyl)phenyl]-1H-benzimidazole-5-(thiazol-2-ylmethyl)-carboxamide

The compound was obtained by reaction with thiazol-2-ylmethylamine in analogy to Example 27c. Yield: 19%, M+H+: 507.

Examples 1, 11 and 17 are intermediates which can be used to prepare the compounds of the formula I.

We claim:

1. A compound of formula I, 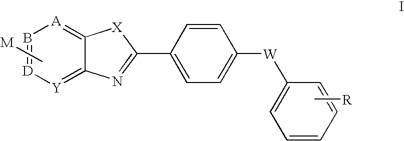
wherein:
M is R1-N(R2)-C(═O)—, R5-C(═O)—N(R1)-, R5-S(O)₀₋₂—N(R1)-, or R1-N(R2)-S(O)₀₋₂—;
W is O, CH₂, or C═O;
X is N—R4, O, or S;
A, B, D and Y are independently of one another C(R3) or N, provided that a maximum of two of the radicals A, B, D, Y are N;
R is (C₁-C₁₆)-alkyl, (C₁-C₅)-alkyloxy, (C₁-C₅)-alkylthio, amino, (C₁-C₅)-alkylamino, di-(C₂-C₈)-alkylamino, (C₁-C₆)-alkylcarbonylamino, (C₁-C₆)-alkoxycarbonylamino, halogen, hydroxy, mono-(C₁-C₆)-alkylaminocarbonyl, di-(C₂-C₈)-alkylaminocarbonyl, (C₁-C₆)-alkoxycarbonyl, (C₁-C₆)-alkylcarbonyl, cyano, trifluoromethyl, trifluoromethyloxy, (C₁-C₆)-alkylsulfonyl or aminosulfonyl;
R1 is (C₂-C₁₀)-alkyl, which is optionally substituted by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino, —(C₆-C₁₀)-aryl, —(C₃-C₁₂)-heteroaryl, —(C₃-C₁₂)-heterocyclyl or —(C₃-C₁₂)-cycloalkyl, wherein the aryl, heteroaryl, heterocyclyl or cycloalkyl is optionally substituted one or more times by halogen, (C₁-C₆)-alkyl, (C₁-C₃)-alkyloxy, hydroxy, (C₁-C₆)-alkylmercapto, amino, (C₁-C₆)-alkylamino, or di-(C₂-C₁₂)-alkylamino,
(C₁-C₁₀)-alkyl, which is substituted by —(C₆-Cₘ)-aryl, —(C₃-C₁₂)-heteroaryl, —(C₃-C₁₂)-heterocyclyl or —(C₃-C₁₂)-cycloalkyl, wherein the aryl, heteroaryl, heterocyclyl or cycloalkyl is optionally substituted one or more times by halogen, (C₁-C₆)-alkyl, (C₁-C₃)-alkyloxy, hydroxy, (C₁-C₆)-alkylmercapto, amino, (C₁-C₆)-alkylamino, or di-(C₂-C₁₂)-alkylamino, or
—(C₆-C₁₀)-aryl, —(C₃-C₁₂)-heteroaryl, —(C₃-C₁₂)-heterocyclyl or —(C₃-C₁₂)-cycloalkyl, Wherein the aryl, heteroaryl, heterocyclyl or cycloalkyl is optionally substituted one or more times by halogen, (C₁-C₆)-alkyl, (C₁-C₃)-alkyloxy, hydroxy, (C₁-C₆)-alkylmercapto, amino, (C₁-C₆)-alkylamino, or di-(C₂-C₁₂)-alkylamino;
R2 is hydrogen, (C₂-C₁₆)-alkyl, —(C₆-C₁₀)-aryl, or (C₁-C₄)-alkylene-(C₆-C₁₀)-aryl; or
R1 and R2 taken together with the nitrogen atom to which they are attached form a monocyclic, saturated or partly unsaturated 4- to 7-membered ring system or a bicyclic saturated or partly unsaturated 8- to 14 membered ring system whose individual members of the ring system may be replaced by one to three atoms or atomic groups from the group of —CHR4-, —CR4R5-, —(C═R4)-, —NR6-, —C(═O)—, —O—, —S—, —SO— and —SO₂—;
R3 is hydrogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino, cyano, (C₁-C₆)-alkylcarbonyl, halogen, trifluoromethyl, trifluoromethyloxy, (C₁-C₆)-alkylsulfonyl, or aminosulfonyl;
R4 is hydrogen, or (C₁-C₅)-alkyl; and
R5 is hydrogen, (C₁-C₁₆)-alkyl, amino, (C₁-C₁₆)-alkylamino, or di-(C₂-C₁₂)-alkylamino, wherein the alkyl is optionally substituted by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino, —(C₆-C₁₀)-aryl, —(C₃-C₁₂)-heteroaryl, —(C₃-C₁₂)-heterocyclyl or (C₃-C₁₂)-cycloalkyl, wherein the aryl, heteroaryl, heterocyclyl or cycloalkyl is optionally substituted one or more times by halogen, (C₁-C₆)-alkyl, (C₁-C₃)-alkyloxy, hydroxy, (C₁-C₆)-alkylmercapto, amino, (C₁-C₆)-alkylamino, or di-(C₂-C₁₂)-alkylamino, or
—(C₆-C₁₀)-aryl, —(C₃-C₁₂)-heteroaryl, —(C₃-C₁₂)-heterocyclyl or —(C₃-C₁₂)-cycloalkyl, wherein the aryl, heteroaryl, heterocyclyl or cycloalkyl is optionally substituted one or more times by halogen, (C₁-C₆)-alkyl, (C₁-C₃)-alkyloxy, hydroxy, (C₁-C₆)-alkylmercapto, amino, (C₁-C₆)-alkylamino, or di-(C₂-C₁₂)-alkylamino;
or a physiologically tolerated salt thereof.

2. The compound according to claim 1, wherein
W is O or C═O;
X is N—R4;
R1 (C₂-C₁₀)-alkyl, which is optionally substituted by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino, —(C₆-C₁₀)-aryl, —(C₃-C₁₂)-heteroaryl, —(C₃-C₁₂)-heterocyclyl or —(C₃-C₁₂)-cycloalkyl, wherein the aryl, heteroaryl, heterocyclyl or cycloalkyl is optionally substituted one or more times by halogen, (C₁-C₆)-alkyl, (C₁-C₃)-alkyloxy, hydroxy, (C₁-C₆)-alkylmercapto, amino, (C₁-C₆)-alkylamino, or di-(C₂-C₁₂)-alkylamino, or
(C₁-C₁₀)-alkyl, which is substituted by —(C₆-C₁₀)-aryl, —(C₃-C₁₂)-heteroaryl, —(C₃-C₁₂)-heterocyclyl or —(C₃-C₁₂)-cycloalkyl, wherein the aryl, heteroaryl, heterocyclyl or cycloalkyl is optionally substituted one or more times by halogen, (C₁-C₆)-alkyl, (C₁-C₃)-alkyloxy, hydroxy, (C₁-C₆)-alkylmercapto, amino, (C₁-C₆)-alkylamino, or di-(C₂-C₁₂)-alkylamino;
R2 is hydrogen, (C₂-C₁₆)-alkyl, —(C₆-C₁₀)-aryl, or (C₁-C₄)-alkylene-(C₆-C₁₀)-aryl;
or R1 and R2 taken together with the nitrogen atom to which they are attached form a monocyclic, saturated or partly unsaturated 4- to 7-membered ring system or a bicyclic saturated or partly unsaturated 8- to 14 membered ring system whose individual members of the ring systems may be replaced by one to three atoms or atomic groups from the group of —CHR4-, —CR4R5-, —(C═R4)-, —NR6-, —C(═O)—, —O—, —S—, —SO— and —SO₂—; and
R5 is hydrogen, (C₁-C₁₆)-alkyl, amino, (C₁-C₁₆)-alkylamino, or di-(C₂-C₁₂)-alkylamino, wherein the alkyl is optionally substituted by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino, —(C₆-C₁₀)-aryl, —(C₃-C₁₂)-heteroaryl, —(C₃-C₁₂)-heterocyclyl or —(C₃-C₁₂)-cycloalkyl, wherein the aryl, heteroaryl, heterocyclyl or cycloalkyl may optionally be substituted one or more times by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino;
or a physiologically tolerated salt thereof.

3. The compound according to claim 1, wherein
M is R1-N(R2)-C(═O)— or R5-C(═O)—N(R1)-;
W is O or C═O;
X is NH;
A, B, D and Y are CH;
R1 is (C₁-C₁₀)-alkyl, which is substituted by —(C₆-C₁₀)-aryl, —(C₃-C₁₂)-heteroaryl, —(C₃-C₁₂)-heterocyclyl or —(C₃-C₁₂)-cycloalkyl, wherein the aryl, heteroaryl, heterocyclyl or cycloalkyl is optionally substituted one or more times by halogen, (C₁-C₆)-alkyl, (C₁-C₃)-alkyloxy, hydroxy, (C₁-C₆)-alkylmercapto, amino, (C₁-C₆)-alkylamino, or di-(C₂-C₁₂)-alkylamino; and
R5 is hydrogen, (C₁-C₁₆)-alkyl, amino, (C₁-C₁₆)-alkylamino, or di-(C₂-C₁₂)-alkylamino, wherein the alkyl is substituted by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino, —(C₆-C₁₀)-aryl, —(C₃-C₁₂)-heteroaryl, —(C₃-C₁₂)-heterocyclyl or —(C₃-C₁₂)-cycloalkyl, wherein the aryl, heteroaryl, heterocyclyl or cycloalkyl is optionally substituted one or more times by halogen, (C₁-C₆)-alkyl, (C₁-C₃)-alkyloxy, hydroxy, (C₁-C₆)-alkylmercapto, amino, (C₁-C₆)-alkylamino, or di-(C₂-C₁₂)-alkylamino;
or a physiologically tolerated salt thereof.

4. The compound according to claim 1, wherein
M is R1-NH—C(═O)—;
W is O or C═O;
X is NH;
A, B, D and Y are CH;
R1 is (C₁-C₁₀)-alkyl, which is substituted by —(C₆-C₁₀)-aryl, —(C₃-C₁₂)-heteroaryl, —(C₃-C₁₂)-heterocyclyl or —(C₃-C₁₂)-cycloalkyl, wherein the aryl, heteroaryl, heterocyclyl or cycloalkyl is optionally substituted one or more times by halogen, (C₁-C₆)-alkyl, (C₁-C₃)-alkyloxy, hydroxy, (C₁-C₆)-alkylmercapto, amino, (C₁-C₆)-alkylamino, or di-(C₂-C₁₂)-alkylamino;
or a physiologically tolerated salt thereof

5. The compound according to claim 1, wherein
M is R1-NH—C(═O)—;
W is O or C═O;
X is NH;
A, B, D and Y are CH;
R1 is —(CH₂), —(C₆-C₁₀)-aryl, —(CH₂)ₙ—(C₃-C₁₂)-heteroaryl, —(CH₂), —(C₃-C₁₂)-heterocyclyl or —(CH₂)ₙ—(C₃-C₁₂)-cycloalkyl, wherein the aryl, heteroaryl, heterocyclyl or cycloalkyl rings is optionally substituted one or more times by halogen, (C₁-C₆)-alkyl, (C₁-C₃)-alkyloxy, hydroxy, (C₁-C₆)-alkylmercapto, amino, (C₁-C₆)-alkylamino, or di-(C₂-C₁₂)-alkylamino; and
n is 1, 2, 3 or 4;
or a physiologically tolerated salt thereof.

6. The compound according to claim 1, which is:
2-(4-Phenoxyphenyl)-3H-benzimidazole-5-(3-pyridin-3-ylpropyl)carboxamide;
2-(4-Phenoxyphenyl)-3H-benzimidazole-5-(3-phenylpropyl)carboxamide;
2-(4-Phenoxyphenyl)-3H-benzimidazole-5-phenethylcarboxamide;
2-(4-Phenoxyphenyl)-3H-benzimidazole-5-(3-methylbutyl)carboxamide;
2-(4-Phenoxyphenyl)-3H-benzimidazole-5-(2-cyclopropylethyl)carboxamide;
2-(4-Phenoxyphenyl)-3H-benzimidazole-5-cyclopropylmethylcarboxamide;
2-(4-Phenoxyphenyl)-3H-benzimidazole-5-(5-chlorothiophen-2-ylmethyl)carboxamide;
2-(4-Phenoxyphenyl)-3H-benzimidazole-5-(thiazol-2-ylmethyl)carboxamide;
2-(4-Phenoxyphenyl)-3H-benzimidazole-5-(3-butoxypropyl)carboxamide;
2-[4-(2-Chlorophenoxy)phenyl]-3H-benzoimidazole-5-phenethylcarboxamide;
2-[4-(2-Chlorophenoxy)phenyl]-3H-benzoimidazole-5-(3-methylbutyl)carboxamide;
2-[4-(2-Chlorophenoxy)phenyl]-3H-benzoimidazole-5-(2-cyclopropylethyl)carboxamide;
2-[4-(2-Chlorophenoxy)phenyl]-3H-benzoimidazole-5-(5-chlorothiophen-2-yl-methyl)carboxamide;
2-[4-(2-Chlorophenoxy)phenyl]-3H-benzoimidazole-5-(thiazol-2-ylmethyl)carboxamide;
2-[4-(2-Trifluoromethylphenoxy)phenyl]-3H-benzimidazole-5-(5-chlorothiophen-2-ylmethyl)-carboxamide;
2-[4-(2-Trifluoromethylbenzoyl)phenyl]-3H-benzimidazole-5-(thiophen-2-ylmethyl)-carboxamide;
2-[4-(2-Trifluoromethylbenzoyl)phenyl]-3H-benzoimidazole-5-isobutylcarboxamide;
2-[4-(2-Trifluoromethylphenoxy)phenyl]-3H-benzimidazole-5-(2-cyclopropylethyl)-carboxamide;
2-[4-(2-Trifluoromethylphenoxy)phenyl]-3H-benzimidazole-5-(3-methylbutyl)carboxamide;
2-[4-(2-Trifluoromethylphenoxy)phenyl]-3H-benzimidazole-5-(thiazol-2-ylmethyl)carboxamide;
2-[4-(2-Trifluoromethylphenoxy)phenyl]-3H-benzimidazole-5-(3-phenylpropyl)carboxamide;
2-[4-(2-Trifluoromethylbenzoyl)phenyl]-3H-benzimidazole-5-(5-chlorothiophen-2-ylmethyl)-carboxamide; or
2-[4-(2-Trifluoromethylbenzoyl)phenyl]-1H-benzimidazole-5-(thiazol-2-ylmethyl)-carboxamide;
or a physiologically tolerated salt thereof.

7. A pharmaceutical composition comprising the compound according to claim 1 or a physiologically tolerated salt thereof, in combination with a pharmacologically acceptable carrier or excipient.

8. The pharmaceutical composition according to claim 6, further comprising one or more active ingredients selected from the group consisting of antidiabetics, hypoglycemic active ingredients, HMGCoA reductase inhibitors, cholesterol absorption inhibitors, PPAR gamma agonists, PPAR alpha agonists, PPAR alpha/gamma agonists, PPAR delta agonists, fibrates, MTP inhibitors, bile acid absorption inhibitors, MTP inhibitors, CETP inhibitors, polymeric bile acid adsorbents, LDL receptor inducers, ACAT inhibitors, antioxidants, lipoprotein lipase inhibitors, ATP-citrate lyase inhibitors, squalene synthetase inhibitors, lipoprotein(a) antagonists, HM74A receptor agonists, lipase inhibitors, insulins, sulfonylureas, biguanides, meglitinides, thiazolidinediones, α-glucosidase inhibitors, active ingredients which act on the ATP-dependent potassium channel of the beta cells, glycogen phosphorylase inhibitors, glucagon receptor antagonists, activators of glucokinase, inhibitors of gluconeogenesis, inhibitors of fructose-1,6-bisphosphatase, modulators of glucose transporter 4, inhibitors of glutamine-fructose-6-phosphate amidotransferase, inhibitors of dipeptidylpeptidase IV, inhibitors of 11-beta-hydroxysteroid dehydrogenase 1, inhibitors of protein tyrosine phosphatase 1B, modulators of the sodium-dependent glucose transporter 1 or 2, modulators of GPR40, inhibitors of hormone-sensitive lipase, inhibitors of acetyl-CoA carboxylase, inhibitors of phosphoenolpyruvate carboxykinase, inhibitors of glycogen synthase kinase-3 beta, inhibitors of protein kinase C beta, endothelin-A receptor antagonists, inhibitors of I kappaB kinase, modulators of the glucocorticoid receptor, CART agonists, NPY agonists, MC4 agonists, orexin agonists, H3 agonists, TNF agonists, CRF agonists, CRF BP antagonists, urocortin agonists, β3 agonists, CB1 receptor antagonists, melanocyte-stimulating hormone agonists, CCK agonists, serotonin reuptake inhibitors, mixed serotoninergic and noradrenergic compounds, 5HT agonists, bombesin agonists, galanin antagonists, growth hormones, growth hormone-releasing compounds, TRH agonists, uncoupling protein 2 or 3 modulators, leptin agonists, DA agonists, bromocriptine, Doprexin, lipase/amylase inhibitors, PPAR modulators, RXR modulators, TR-β agonists and amphetamines.

9. A pharmaceutical composition comprising the compound according to claim 2 or a physiologically tolerated salt thereof, in combination with a pharmacologically acceptable carrier or excipient.

10. A pharmaceutical composition comprising the compound according to claim 3 or a physiologically tolerated salt thereof, in combination with a pharmacologically acceptable carrier or excipient.

11. A pharmaceutical composition comprising the compound according to claim 4 or a physiologically tolerated salt thereof, in combination with a pharmacologically acceptable carrier or excipient.

12. A pharmaceutical composition comprising the compound according to claim 5 or a physiologically tolerated salt thereof, in combination with a pharmacologically acceptable carrier or excipient.

13. A pharmaceutical composition comprising the compound according to claim 6 or a physiologically tolerated salt thereof, in combination with a pharmacologically acceptable carrier or excipient.

14. A method for treating obesity, diabetes, metabolic syndrome, insulin resistance cardiovascular disorder, CNS disorder, schizophrenia, Alzheimer's disease, or for lowering lipid level, in a patient in need thereof, comprising administering to the patient a pharmaceutically effective amount of the compound according to claim 1 or a physiologically tolerated salt thereof.

15. A process for preparing a pharmaceutical composition comprising the compound according to claim 1 or a physiologically tolerated salt thereof, in combination with a pharmacologically acceptable carrier or excipient, comprising mixing the compound according to claim 1 or the physiologically compatible salt thereof with the pharmacologically acceptable carrier or excipient, and converting the mixture into to a form suitable for administration.

16. A method for treating obesity, diabetes, metabolic syndrome, insulin resistance cardiovascular disorder, CNS disorder, schizophrenia, Alzheimer's disease, or for lowering lipid level, in a patient in need thereof, comprising administering to the patient a pharmaceutically effective amount of a compound of formula I 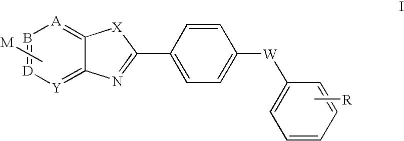
wherein:
M is R1-N(R2)-C(═O)—, R5-C(═O)—N(R1)-, R5-S(O)₀₋₂—N(R1)-, R1-N(R2)-S(O)₀₋₂—, or a heterocycle which may comprise 2 to 4 heteroatoms from the group of N, O and S, wherein the heterocycle is optionally substituted by (C₁-C₁₆)-alkyl, or doubly bonded oxygen;
W is O, CH₂ or C═O;
X is N—R4, O or S;
A, B, D and Y are independently of one another C(R3) or N, provided that a maximum of two of the radicals A, B, D, Y are N;
R is hydrogen, (C₁-C₁₆)-alkyl, (C₁-C₅)-alkyloxy, (C₁-C₅)-alkylthio, amino, (C₁-C₅)-alkylamino, di-(C₂-C₈)-alkylamino, (C₁-C₆)-alkylcarbonylamino, (C₁-C₆)-alkoxycarbonylamino, halogen, hydroxy, mono-(C₁-C₆)-alkylaminocarbonyl, di-(C₂-C₈)-alkylaminocarbonyl, (C₁-C₆)-alkoxycarbonyl, (C₁-C₆)-alkylcarbonyl, cyano, trifluoromethyl, trifluoromethyloxy, (C₁-C₆)-alkylsulfonyl or aminosulfonyl;
R1 is (C₂-C₁₀)-alkyl, which is optionally substituted by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino, —(C₆-C₁₀)-aryl, —(C₃-C₁₂)-heteroaryl, —(C₃-C₁₂)-heterocyclyl or —(C₃-C₁₂)-cycloalkyl, wherein the aryl, heteroaryl, heterocyclyl or cycloalkyl is optionally substituted one or more times by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino,
(C₁-C₁₀)-alkyl, which is substituted by —(C₆-C₁₀)-aryl, —(C₃-C₁₂)-heteroaryl, —(C₃-C₁₂)-heterocyclyl or —(C₃-C₁₂)-cycloalkyl, wherein the aryl, heteroaryl, heterocyclyl or cycloalkyl is optionally substituted one or more times by halogen, (C₁-C₆)-alkyl, (C₁-C₃)-alkyloxy, hydroxy, (C₁-C₆)-alkylmercapto, amino, (C₁-C₆)-alkylamino, or di-(C₂-C₁₂)-alkylamino, or
—(C₆-C₁₀)-aryl, —(C₃-C₁₂)-heteroaryl, —(C₃-C₁₂)-heterocyclyl or —(C₃-C₁₂)-cycloalkyl, wherein the aryl, heteroaryl, heterocyclyl or cycloalkyl is optionally substituted one or more times by halogen, (C₁-C₆)-alkyl, (C₁-C₃)-alkyloxy, hydroxy, (C₁-C₆)-alkylmercapto, amino, (C₁-C₆)-alkylamino, or di-(C₂-C₁₂)-alkylamino;
R2 is hydrogen, (C₂-C₁₆)-alkyl, —(C₆-C₁₀)-aryl, or (C₁-C₄)-alkylene-(C₆-C₁₀)-aryl;
or R1 and R2 taken together with the nitrogen atom to which they are attached form a monocyclic, saturated or partly unsaturated 4- to 7-membered ring system or a bicyclic saturated or partly unsaturated 8- to 14 membered ring system whose individual members of the ring system may be replaced by one to three atoms or atomic groups from the group of —CHR4-, —CR4R5-, —(C═R4)-, —NR6-, —C(═O)—, —O—, —S—, —SO— and —SO₂—;
R3 is hydrogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino, cyano, (C₁-C₆)-alkylcarbonyl, halogen, trifluoromethyl, trifluoromethyloxy, (C₁-C₆)-alkylsulfonyl, or aminosulfonyl;
R4 is hydrogen or (C₁-C₅)-alkyl; and
R5 is hydrogen, (C₁-C₁₆)-alkyl, amino, (C₁-C₁₆)-alkylamino, or di-(C₂-C₁₂)-alkylamino, wherein the alkyl is optionally substituted by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino, —(C₆-C₁₀)-aryl, —(C₃-C₁₂)-heteroaryl, —(C₃-C₁₂)-heterocyclyl or —(C₃-C₁₂)-cycloalkyl, wherein the aryl, heteroaryl, heterocyclyl or cycloalkyl is optionally substituted one or more times by halogen, (C₁-C₆)-alkyl, (C₁-C₃)-alkyloxy, hydroxy, (C₁-C₆)-alkylmercapto, amino, (C₁-C₆)-alkylamino, or di-(C₂-C₁₂)-alkylamino, or
—(C₆-C₁₀)-aryl, —(C₃-C₁₂)-heteroaryl, —(C₃-C₁₂)-heterocyclyl or —(C₃-C₁₂)-cycloalkyl, wherein the aryl, heteroaryl, heterocyclyl or cycloalkyl is optionally substituted one or more times by halogen, (C₁-C₆)-alkyl, (C₁-C₃)-alkyloxy, hydroxy, (C₁-C₆)-alkylmercapto, amino, (C₁-C₆)-alkylamino, or di-(C₂-C₁₂)-alkylamino;
or a physiologically tolerated salt thereof.

17. The method according to claim 16, wherein:
W is O or C═O;
X is N—R4;
R1 is (C₂-C₁₀)-alkyl, which is optionally substituted by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino, —(C₆-C₁₀)-aryl, —(C₃-C₁₂)-heteroaryl, —(C₃-C₁₂)-heterocyclyl or —(C₃-C₁₂)-cycloalkyl, wherein the aryl, heteroaryl, heterocyclyl or cycloalkyl is optionally substituted one or more times by halogen, (C₁-C₆)-alkyl, (C₁-C₃)-alkyloxy, hydroxy, (C₁-C₆)-alkylmercapto, amino, (C₁-C₆)-alkylamino, or di-(C₂-C₁₂)-alkylamino, or
(C₁-C₁₀)-alkyl, which is substituted by —(C₆-C₁₀)-aryl, —(C₃-C₁₂)-heteroaryl, —(C₃-C₁₂)-heterocyclyl or —(C₃-C₁₂)-cycloalkyl, wherein the aryl, heteroaryl, heterocyclyl or cycloalkyl is optionally substituted one or more times by halogen, (C₁-C₆)-alkyl, (C₁-C₃)-alkyloxy, hydroxy, (C₁-C₆)-alkylmercapto, amino, (C₁-C₆)-alkylamino, or di-(C₂-C₁₂)-alkylamino;
R2 is hydrogen, (C₂-C₁₆)-alkyl, —(C₆-C₁₀)-aryl, or (C₁-C₄)-alkylene-(C₆-C₁₀)-aryl;
or R1 and R2 taken together with the nitrogen atom to which they are attached form a monocyclic, saturated or partly unsaturated 4- to 7-membered ring system or a bicyclic saturated or partly unsaturated 8- to 14 membered ring system whose individual members of the ring systems may be replaced by one to three atoms or atomic groups from the group of —CHR4-, —CR4R5-, —(C═R4)-, —NR6-, —C(═O)—, —O—, —S—, —SO— and —SO₂—; and
R5 is hydrogen, (C₁-C₁₆)-alkyl, amino, (C₁-C₁₆)-alkylamino, or di-(C₂-C₁₂)-alkylamino,
wherein the alkyl is optionally substituted by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino, —(C₆-C₁₀)-aryl, —(C₃-C₁₂)-heteroaryl, —(C₃-C₁₂)-heterocyclyl or —(C₃-C₁₂)-cycloalkyl, wherein the aryl, heteroaryl, heterocyclyl or cycloalkyl is optionally substituted one or more times by halogen, (C₁-C₆)-alkyl, (C₁-C₃)-alkyloxy, hydroxy, (C₁-C₆)-alkylmercapto, amino, (C₁-C₆)-alkylamino, or di-(C₂-C₁₂)-alkylamino;
or a physiologically tolerated salt thereof.

18. The method according to claim 16, wherein:
M is R1-N(R2)-C(═O)— or R5-C(═O)—N(R1)-;
W is O or C═O;
X is N—R4;
R1 is (C₂-C₁₀)-alkyl, which is optionally substituted by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino, —(C₆-C₁₀)-aryl, —(C₃-C₁₂)-heteroaryl, —(C₃-C₁₂)-heterocyclyl or —(C₃-C₁₂)-cycloalkyl, wherein the aryl, heteroaryl, heterocyclyl or cycloalkyl may optionally be substituted one or more times by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino;
(C₁-C₁₀)-alkyl, which is substituted by —(C₆-C₁₀)-aryl, —(C₃-C₁₂)-heteroaryl, —(C₃-C₁₂)-heterocyclyl or —(C₃-C₁₂)-cycloalkyl, wherein the aryl, heteroaryl, heterocyclyl or cycloalkyl is optionally substituted one or more times by halogen, (C₁-C₆)-alkyl, (C₁-C₃)-alkyloxy, hydroxy, (C₁-C₆)-alkylmercapto, amino, (C₁-C₆)-alkylamino, or di-(C₂-C₁₂)-alkylamino;
R2 is hydrogen, (C₂-C₁₆)-alkyl, —(C₆-C₁₀)-aryl, or (C₁-C₄)-alkylene-(C₆-C₁₀)-aryl;
or R1 and R2 taken together with the nitrogen atom to which they are attached form a monocyclic, saturated or partly unsaturated 4- to 7-membered ring system or a bicyclic saturated or partly unsaturated 8- to 14 membered ring system whose individual members of the ring systems may be replaced by one to three atoms or atomic groups from the group of —CHR4-, —CR4R5-, —(C═R4)-, —NR6-, —C(═O)—, —O—, —S—, —SO— and —SO₂—; and
R5 is hydrogen, (C₁-C₁₆)-alkyl, amino, (C₁-C₁₆)-alkylamino, or di-(C₂-C₁₂)-alkylamino, wherein the alkyl is optionally substituted by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino, —(C₆-C₁₀)-aryl, —(C₃-C₁₂)-heteroaryl, —(C₃-C₁₂)-heterocyclyl or —(C₃-C₁₂)-cycloalkyl, wherein the aryl, heteroaryl, heterocyclyl or cycloalkyl is optionally substituted one or more times by halogen, (C₁-C₆)-alkyl, (C₁-C₃)-alkyloxy, hydroxy, (C₁-C₆)-alkylmercapto, amino, (C₁-C₆)-alkylamino, or di-(C₂-C₁₂)-alkylamino;
or a physiologically tolerated salt thereof.

19. The method according to claim 17, wherein:
M is R1-N(R2)-C(═O)— or R5-C(═O)—N(R1)-;
W is O or C═O;
X is N—R4;
A, B, D and Y are CH;
R1 is (C₂-C₁₀)-alkyl, which is optionally substituted by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino, —(C₆-C₁₀)-aryl, —(C₃-C₁₂)-heteroaryl, —(C₃-C₁₂)-heterocyclyl or (C₃-C₁₂)-cycloalkyl, wherein the aryl, heteroaryl, heterocyclyl or cycloalkyl is optionally substituted one or more times by halogen, (C₁-C₆)-alkyl, (C₁-C₃)-alkyloxy, hydroxy, (C₁-C₆)-alkylmercapto, amino, (C₁-C₆)-alkylamino, or di-(C₂-C₁₂)-alkylamino, or
(C₁-C₁₀)-alkyl, which is substituted by —(C₆-C₁₀)-aryl, —(C₃-C₁₂)-heteroaryl, —(C₃-C₁₂)-heterocyclyl or —(C₃-C₁₂)-cycloalkyl, wherein the aryl, heteroaryl, heterocyclyl or cycloalkyl is optionally substituted one or more times by halogen, (C₁-C₆)-alkyl, (C₁-C₃)-alkyloxy, hydroxy, (C₁-C₆)-alkylmercapto, amino, (C₁-C₆)-alkylamino, or di-(C₂-C₁₂)-alkylamino;
R2 is hydrogen, (C₂-C₁₆)-alkyl, —(C₆-C₁₀)-aryl, or (C₁-C₄)-alkylene-(C₆-C₁₀)-aryl;
or R1 and R2 taken together with the nitrogen atom to which they are attached form a monocyclic, saturated or partly unsaturated 4- to 7-membered ring system or a bicyclic saturated or partly unsaturated 8- to 14 membered ring system whose individual members of the ring systems may be replaced by one to three atoms or atomic groups from the group of —CHR4-CR4R5-, —(C═R4)-, —NR6-, —C(═O)—, —O—, —S—, —SO— and —SO₂—;
R4 is hydrogen; and
R5 is hydrogen, (C₁-C₁₆)-alkyl, amino, (C₁-C₁₆)-alkylamino, or di-(C₂-C₁₂)-alkylamino, wherein the alkyl is optionally substituted by halogen, (C₁-C₆)-alkyl, (C₁-C₃)-alkyloxy, hydroxy, (C₁-C₆)-alkylmercapto, amino, (C₁-C₆)-alkylamino, di-(C₂-C₁₂)-alkylamino, —(C₆-C₁₀)-aryl, —(C₃-C₁₂)-heteroaryl, —(C₃-C₁₂)-heterocyclyl or —(C₃-C₁₂)-cycloalkyl, wherein the aryl, heteroaryl, heterocyclyl or cycloalkyl is optionally substituted one or more times by halogen, (C₁-C₆)-alkyl, (C₁-C₃)-alkyloxy, hydroxy, (C₁-C₆)-alkylmercapto, amino, (C₁-C₆)-alkylamino, or di-(C₂-C₁₂)-alkylamino;
or a physiologically tolerated salt thereof.
